(19) United States
(12) Patent Application Publication
(10) Pub. No.: US 2017/0049851 A1
(43) Pub. Date: Feb. 23, 2017

(54) Method for Medical Treatment

(71) Applicant: Richard Postrel, Miami Beach, FL (US)

(72) Inventor: Richard Postrel, Miami Beach, FL (US)

(21) Appl. No.: 15/222,955

(22) Filed: Jul. 29, 2016

Related U.S. Application Data
(60) Provisional application No. 62/198,124, filed on Jul. 29, 2015.

(51) Int. Cl. A61K 38/13; A61K 31/525; A61K 31/198; A61K 31/197; A61K 31/375; A61K 33/32; A61K 33/06; A61K 33/30; A61K 31/4172; A61K 31/355; A61K 31/05; A61K 31/381; A61K 31/519; A61K 31/19; A61K 31/194; A61K 31/5575; A61K 31/616; A61K 31/4045; A61K 31/46; A61K 31/137; A61K 31/655; A61K 38/06; A61K 31/015; G06F 19/00; A61K 31/122

(52) U.S. Cl. CPC A61K 38/13; A61K 31/122; A61K 31/525; A61K 31/198; A61K 31/197; A61K 31/375; A61K 33/32; A61K 33/06; A61K 33/30; A61K 31/4172; A61K 31/355; A61K 31/05; A61K 31/381; A61K 31/519; A61K 31/19; A61K 31/194; A61K 31/5575; A61K 31/616; A61K 31/4045; A61K 31/46; A61K 31/137; A61K 31/655; A61K 38/063; A61K 31/015; G06F 19/345

(57) ABSTRACT

The disclosure provides improved methods for treating or preventing a class of undesired health events including multiple related maladies, such as a disease, condition, or syndrome or the like. The improvement results from optimization of energy metabolism by administering a therapeutically effective compound selected to a) modulate mitochondrial activity to correct for deficiencies resulting from the disease, b) to boost cell energy metabolism thereby improving the original method's efficacy, and/or c) to correct for metabolic disruptions resulting from therapies or medicaments used in the method to be improved. A combination therapy may be designed based on a disease, a group and/or an individual, said combination comprising one or more energy optimization booster combined with a medicament used in an original method is an exemplary embodiment. In some circumstances optimization may involve boosting energy metabolism throughout an organism or in selected cells, tissues or subcellular structures; in some circumstances optimization may involve diminishing energy metabolism throughout an organism or in selected tissues, cells or subcellular structures. In several circumstances diminishing energy metabolism in selected cells to zero or near zero may, by essentially destroying or eliminating mitochondrial functionality of these cells to impair or destroy adverse functionality of these cells or subcellular activity, be optimal for the organism. Biochemicals, including biotherapeutics, may be delivered by any effective method or device, including, but not limited to: injection, oral dosing, nanodelivery, suppository, patches, eye drops, nasal spray, ointment, cream, synthetic gene, conjugated molecule, catheter, timed or controlled release capsule or pill, subdermal implants, diet, etc.

### CROSS REFERENCE TO RELATED APPLICATION

The present application claims benefit to provisional application 62/198,124 filed Jul. 29, 2015.

### FIELD OF THE INVENTION

The claimed subject matter relates generally to the field of disease therapy and, more particularly, to the improved treatment of disease. The improved method optimizes treatment using a therapeutically effective combination of a first therapy known to beneficially modulate disease or disease progression and a second therapy paired with the first therapy that advances or achieves optimal cellular function.

### BACKGROUND

Throughout history mankind has used various therapies to improve quality of life. Early therapies were sometimes accidental or arrived at through a trial and error approach. There is evidence that plant or animal parts or extracts were used in prehistoric time with a beneficially medical effect.

In recent history science has provided understanding of chemistry and biology so that more pure and more precisely targeted therapies have become available.

Mankind has recognized a need for therapeutic intervention, sometimes as simple as proper nutrition. But as therapies are more precisely targeted many functions of the cell have been successfully modulated through treatment to improve cell and individual health.

These therapies are selected because of a desired effect on one or more tissues or to achieve a desired general outcome. However, given the complexities of cell physiology, especially human cell physiology, effects caused by one dynamic modulation almost invariably depend upon or affect other cell functions. Sometimes these dependencies or network effects work in concert to benefit the cell and the individual. But often compromises are made intentionally or unintentionally to beneficially modulate one component or function to the detriment of a second component or function. Or when the therapy is applied, full benefit is not achieved due to a secondary or tertiary co-function. Modern medicine understands tradeoffs involving administering a therapy achieving a desired effect that is often associated with one or more undesired effect, aka, a side effect.

In common practice, often medical therapy is compromised because a patient's metabolism is weakened because of intrinsic or extrinsic factors or because the therapy itself will change normal cell metabolism. Accordingly, the full beneficial effect of the medical therapy is limited by one or more other cell function, in particular cell energy metabolism.

Human cells are eukaryotic cells and therefore, like eukaryotic cells generally, they rely on their mitochondria to produce adenosine triphosphate (ATP). In each mitochondrion at the mitochondrial inner membrane, electrons from NADH and succinate are transported by the Electron Transport Chain (ETC) to oxygen, which, when it accepts the electrons, is reduced to combine with hydrogen to make water. Along the way the ETC comprises several donor and receptor enzymes in series, eventually depositing the electrons with an oxygen. Passing electrons from donor to acceptor releases energy in the form of a proton (H⁺) across the mitochondrial membrane, This ion flux has the potential to do work. This metabolic process is known as oxidative phosphorylation and results in production of adenosine triphosphate, aka, ATP. The mitochondrion is important to cell metabolism and survival. Detailed descriptions are known or can be found in the art.

Thus the mitochondrion organelle is essential for healthy cells and therefore for healthy human life. ATP, an essential molecule for energy metabolism within the cell is primarily generated by mitochondria. Processes such as adaptive thermogenesis, ion homeostasis, immune responses, production of reactive oxygen species, and programmed cell death (apoptosis) are some of the more complex processes that also require appropriate ATP synthesis and transport. Mitochondria contain their own DNA (mtDNA), which serves as a template for 13 mitochondrial proteins, 2 ribosomal RNAs (rRNAs), and 22 transfer RNAs (tRNAs). However, the mitochondrion can not function as a distinct and independent organelle. Replication, transcription, translation, and repair of mtDNA require proteins encoded by nuclear DNA (nDNA) of the hosting cell.

Modern mitochondria have many similarities to some modern prokaryotes, even though they have diverged significantly from the early prokaryotes since the ancient symbiotic event. For example, the inner mitochondrial membrane contains electron transport proteins like the plasma membrane of prokaryotes, and mitochondria also have their own prokaryote-like circular genome. But one difference is that these organelles are thought to have “lost” most of the genes once carried by their prokaryotic ancestor. Although present-day mitochondria do synthesize a few of their own proteins, a vast majority of the proteins they require to maintain the host cell are now encoded in the nuclear genome of the host.

Thus mitochondrial based abnormalities or dysfunctions may have mitochondrial or cellular origination. And a disturbance or dysfunction of any of the related pathways can compromise mitochondrial function, cellular energy metabolism and accordingly, health of the entire organism, such as a human.

Mitochondrial dysfunction is observed in some monogenic mitochondrial disorders, but is also associated with many more common and abstract (not attributable to a single genetic defect) pathologic conditions, such as Alzheimer's disease, Parkinson's disease, cancer, cardiac disease, diabetes, epilepsy, Huntington's disease, and obesity.

While several researchers have investigated treatment to overcome primary dysfunction of mitochondria, there is still a need for improvement in this area. But, more importantly, improvement of mitochondrial function to augment treatment of diseases primarily resulting from other, i.e., non-mitochondrial, based sources, be they intrinsic to the organism or extrinsic (e.g., from toxins or other exposures) has not been addressed. The present invention starts filling this void with, for example, provision of a pharmaceutical composition that combines a treatment for the primary malady with one or more compounds designed to optimize mitochondrial performance. A method to select the optimizing composition for the disease, the stage of the disease, the genetic of physiologic background of the individual and ideally for the individual at the precise time of treatment is also included in embodiments of this invention By optimizing mitochondrial performance (function) the pharmaceutical composition provides that the treatment for the primary malady can have strengthened beneficial effect.

### DETAILED DESCRIPTION

In general words in this description will have a meaning as used in American English. The following list is provided as additional guidance.

### DEFINITIONS

ADP—adenosine diphosphate. Higher ADP levels are often associated with higher respiratory activity.

ATP—adenosine triphosphate, a primary molecule involved in energy storage, transport and release.

Biogenesis—a synthetic process occurring as part of metabolism in a living organism.

Cellular metabolism—set of chemical reactions that occurs in living organisms to maintain life. Metabolism includes both anabolism and catabolism as well as multiple pathways that maintain life functions within a cell or organism. The cellular metabolism of an individual cell or cell type may be optimized with respect to the whole organism which may involve boosting or diminishing metabolism in a selected cell. There is no real count of an actual number of metabolic pathways. With branches and cycles within major pathways and pathways sometimes only active in specific cell types and sometimes only at select times, counting an actual number would be arbitrary. However, the skilled artisan appreciates that the total number of pathways, including subpaths numbers in the thousands. The internet is an available resource to study classes of pathways or individual pathways. See e.g., wwwitsokaytobesmart.com, though there are many webpages available relating to metabolic pathways.
Boosted cellular metabolism—cellular metabolism altered in a manner to increase activity of one or more desired pathways of that cell. Desired pathways may be different depending on cell type and status of the organism where the cell resides. For example, shifting from anaerobic pathway to aerobic pathway will in most circumstances be considered a boost.
Diminished cellular metabolism—cellular metabolism altered in a manner to decrease activity of one or more desired pathways of that cell. For example, if a tissue is producing a substance in excess of the organisms needs or in an amount slowing production of a more needed substance diminishing metabolism in the overproducing cells may allow the organism to thrive. In some examples, decreasing metabolism to zero thereby causing elimination of a class of cells, e.g., a cancerous class of cells may be of great benefit to the organism. Dosage of one or more medicaments might be one means of managing and fine-tuning metabolic levels and mitochondrial functionality. In certain cases such as with cancer it may be desirous to target the cancerous cells more directly, for example targeting a receptor or genomic profile specific to or overabundant in the cancerous cell. By diminishing cellular metabolism in these cells, subcellular structures or regions to levels reaching or approaching zero, an entire class of cells may lose functionality or be eliminated.

Clinical improvement—An observable improvement in at least one factor in a patient's quality of life.

Coenzyme Q (CoQ₁₀)—aka: ubiquinone or ubidecarenone. An oil-soluble, vitamin-like substance is present in mitochondria. CoQ₁₀ is part of the electron transport chain participating in aerobic cellular respiration to form ATP. CoQ₁₀ is especially significant because of its respiratory functions and because cholesterol inhibitors, such as statins can also inhibit synthesis of CoQ₁₀ precursors.

Desmin—An intermediate filament (IF) protein expressed in striated and smooth muscle tissues and is one of the earliest known muscle-specific genes to be expressed during cardiac and skeletal muscle development. Desmin is seen as controlling mitochondrial function by interaction with myofibrils and interacting with the cytoskeleton to affect positioning within a cell.

ETC—electron transport chain which is used to harvest energy for use in metabolism.

Kcnq2—a member of the kcnq family of proteins which act as ion channels controlling potassium (K) flux across membranes. Potassium gradients can control electrical potential across a membrane and therefore can be involved with electrical signaling within and between cells. A potassium gradient can also control flux of other ions.

kif5b and kif5b—a gene encoding the protein and the encoded a heavy chain portion of kif5 protein working through microtubules to effect appropriate distribution of mitochondria within a cell. Mitochondria are not its only cargo; the protein is also associated with lysozyme and endocytic vessel distribution and is an essential component for distribution of many proteins within a cell. Neurons also express related proteins encoded by kif5a and kif5c.

Mitochondrial integrity—Mitochondrial integrity is known as a controlling factor in apoptosis, cell controlled self-destruction. Integrity may be comprised by events including, but not limited to: membrane permeability changes, altered exposure of membrane proteins, changed expression of the mitochondrial genome.

Mitochondrial protein—a protein encoded by or used within a mitochondrion.

Mitochondrial supportive substance—a chemical that changes mitochondrial activity to benefit at least one aspect of cellular metabolism.

mtDNA—double-stranded DNA found exclusively in mitochondria that in most eukaryotes is a circular molecule. A single mitochondrion may include multiple copies of this circular mtDNA molecule.

Optimization—As used herein, optimization has the general meaning of a process leading to an improved outcome. Optimization will generally incorporate at least one facet of enhancement of number, outcome function or the like. In some uses optimization may refer to maximizing a component or process or a selected group of components and/or processes. More loosely optimization is used to mean improvement, even if a greater improvement might be obtainable. Many factors and outcomes, including but not limited to: effect on other processes, availability of an instrument, component or professional, cost, location, patient's wishes and government regulation may be factors in the optimization procedure and ultimate decisions made to determine a level of optimization. Optimization for one patients often will differ from optimization for another patient, but each patient will have improvement. Optimization may often involve improving one or more outcomes in concert with a possible worsening of another component of process.

Optimizing—The process of optimization. Optimization or the process is considered as a goal or a work in progress approaching an optimal or best outcome. Thus optimization may vary with time.

Organic—a compound containing carbon. A molecule having carbon and at least one other element.

Plectin—A protein found in several isoforms that is ubiquitous in the cytoskeleton of most mammalian cells. Plectin links actin microfilaments, microtubules and intermediate filaments (IF) together. Plectin also appears outside the cell in the extracellular linkages between cells.

Restore—to bring something to or towards a previous condition, a normal condition or an improved condition. The condition may be defined as a number or concentration, a rate of activity, a structure, or any observable or measurable process or product of metabolism.

Vimentin—VimIF, an intermediate filament protein that is involved in distribution, motility and anchoring of mitochondria. Vimentin can work with dyneins and actin-dependent myosins within the cell to deliver and anchor mitochondria close to where metabolic requirements are high.

### The Mitochondrion: Optimization Target 1

Mitochondria, one of the organelles found in most eukaryotic cells are often called the “powerhouse” or “battery” of the cell. A eukaryotic cell typically has multiple mitochondria, the number being higher in cells with higher metabolisms. The molecule adenosine triphosphate (ATP) functions as a predominant energy carrier in the cell. Eukaryotic cells derive the majority of their ATP from biochemical processes carried out by their mitochondria. Within the cell mitochondria also tend to be found in regions with higher activities. Each cell has mechanisms to control mitochondrial synthesis and degradation and by balancing these mechanisms can control the number of mitochondria and metabolic rate of the cell. Cells also control movement of mitochondria so that their substrates and products can be efficiently delivered. Assisting the cells and organism containing the cells to optimize these activities will be found valuable in optimizing therapeutic outcomes.

These biochemical processes carried out by mitichondria include, but are not limited to the following important cycles: i) the citric acid cycle (the tricarboxylic acid cycle, or Kreb's cycle), generating reduced nicotinamide adenine dinucleotide (NADH+H+) from oxidized nicotinamide adenine dinucleotide (NAD+), and ii) oxidative phosphorylation, during which NADH+H⁺ is oxidized back to NAD⁺. (The citric acid cycle also reduces flavin adenine dinucleotide, or FAD, to FADH2; FADH2 also participates in oxidative phosphorylation.)

The respiratory chain of a mitochondrion is located in the inner mitochondrial membrane and consists of five multimeric protein complexes: Complex I; (approximately 44 subunits), Complex II (approximately 4 subunits), Complex III (approximately 11 subunits), Complex IV (approximately 13 subunits) and Complex V (approximately 16 units). (The reported number of subunits is given as approximate because the counts are different in different reports due to improving scientific understanding.) The respiratory chain also requires two small electron carriers, ubiquinone (coenzyme Q1) and cytochrome c.

ATP synthesis involves two coordinated processes: 1) electrons are transported horizontally from complexes I and II to coenzyme Q to Complex III to cytochrome c to Complex IV, and ultimately to the final electron acceptor, molecular oxygen, thereby producing water. At the same time, protons are pumped “vertically” across the mitochondrial inner membrane (i.e., from the matrix to the inter membrane space) by complexes I, II, II and IV. ATP is generated by the influx of these protons back into the mitochondrial matrix through complex V (mitochondrial ATP synthase). The energy released as these electrons traverse the complexes is used to generate a proton gradient across the inner membrane of the mitochondrion, which results in stored potential energy in the form of an electrochemical potential across the inner membrane.

In this process, Complex I (NADH dehydrogenase, also called NADH:ubiquinone oxidoreductase) removes two electrons from NADH and transfers them to a lipid-soluble carrier, ubiquinone. The reduced product, ubiquinol, is free to diffuse within the membrane.

At the same time, Complex I moves four protons (H⁺) across the membrane, producing a proton gradient. Complex I is one of the main sites at which premature electron leakage to oxygen occurs, thus being one of main sites of production of one harmful free radical called superoxide.

Complex II (succinate dehydrogenase) funnels additional electrons into the quinone pool by removing electrons from succinate and transferring them (via FAD) to the quinone pool.

Complex II consists of four protein subunits: SDHA, SDHB, SDHC, and SDHD. Other electron donors (e.g., fatty acids and glycerol 3-phosphate) also funnel electrons into the quinone pool (via FAD), again without producing a proton gradient.

Complex III (cytochrome b/c complex) removes two electrons from QH₂ and transfers them to two molecules of cytochrome c, the water-soluble electron carrier located between the membranes. As part of this process, it moves two protons across the membrane, producing a proton gradient (in total 4 protons: 2 protons are translocated and 2 protons are released from ubiquinol). When electron transfer is hindered (e.g., by a high membrane potential, point mutations or by respiratory inhibitors such as antimycin A), Complex III can leak electrons to oxygen resulting in the formation of superoxide, a highly-toxic oxidative species, which appears in the pathology of many diseases and is seen in aging.

Complex IV (cytochrome c oxidase) removes four electrons from four molecules of cytochrome c and transfers them to molecular oxygen (O₂), producing two molecules of water (H₂O). At the same time, it moves four protons (H⁺) across the membrane, producing a proton gradient. Complex V (mitochondrial ATP synthetase) which is not directly associated with Complexes I, II, III and IV uses the energy stored by the electrochemical proton gradient to convert ADP into ATP.

McCormack et al. (2012) characterized one facet of mitochondrial disease as follows:
Mitochondrial respiratory chain disease is an increasingly well-recognized, but notoriously heterogeneous, group of multisystemic energy deficiency disorders. Its extensive heterogeneity has presented a substantial obstacle for establishing a definitive genetic diagnosis and clear pathogenic understanding in individual patients with suspected mitochondrial disease. While known genetic causes of “classical” mitochondrial DNA (mtDNA)—based disease syndromes have been readily diagnosable, the overwhelming majority of patients with clinical and/or biochemical evidence of suspected mitochondrial disease have had no identifiable genetic etiology for their debilitating or lethal disease. McCormick et al. 2012

To date about 10³ genes encoding mitochondrial proteins have been identified in humans (MitoCarta human inventory, Broad Institute). Mitochondrial dysfunction can arise from a mutation in one of these genes (causing a primary mitochondrial disorder) or from an outside influence on mitochondria (causing a secondary mitochondrial disorder). Mutations in 228 protein-encoding nDNA genes and 13 mtDNA genes have been linked to a human disorder. The involvement of the activity of these genes in disorders emphasizes that optimizing function of any of these where they are found deficient can improve medical therapy.

Mitochondrial DNA is more prone to mutation effects in that the mitochondrion has a high rate of replication and lacks a DNA repair pathway in the organelle. The high level of active oxygens and the resultant oxidative stress also probably contribute to a relatively rapid mtDNA mutation rate. Thus control of mtDNA mutation and control of mutated mtDNA can be important targets for optimization.

The present invention may target any one or more of these genes, control of these genes, expression products of these in optimization.

Common pharmaceutical drugs such as amiodarone, biguanides, haloperidol, statins, valproic acid, zidovudine, anesthetics, antibiotics, chemotherapeutic agents, and even NSAIDS like aspirin (acetylsalicylic acid) have been observed to affect total mitochondrial function. Given the multiple actions of drugs and their specificities for on and off target action, many drugs may lead more frequently to adverse reactions and side effects in patients with mitochondrial disorders than in otherwise healthy persons.

A recent search of Wikipedia (https://en.wikipedia.org/wiki/Mitochondrial disease) accessed Jul. 7, 2015 and again Jul. 26, 2016 found the teaching:
Mitochondrial diseases are sometimes (about 15% of the time) used by mutations in the mtDNA that affect mitochondrial function. Other causes of mitochondrial disease are mutations in genes of the nDNA, whose gene products are imported into the Mitochondria (Mitochondrial proteins) as well as acquired mitochondrial conditions. Mitochondrial diseases take on unique characteristics both because of the way the diseases are often inherited and because mitochondria are so critical to cell function. The subclass of these diseases that have neuromuscular disease symptoms are often called a mitochondrial myopathy.

Mitochondrial Membranes as Structure

As previously mentioned, mitochondria contain two membranes. The outer mitochondrial membrane encompasses the inner membrane, with a small intermembrane space in between. The outer membrane has many protein-based pores that can allow the passage of simple ions and molecules as large as a small protein. In contrast, the inner membrane has much more restricted permeability. It is more like the plasma membrane of a cell. The inner membrane anchors proteins involved in electron transport and ATP synthesis. This membrane surrounds the mitochondrial matrix (the innermost compartment within the mitochondrion), where the citric acid cycle produces the electrons that travel from one protein complex to the next along the inner membrane. At the end of the ETC, the final electron acceptor is oxygen which ultimately forms water (H₂O). At the same time, the electron transport chain produces ATP. ADP (adenosine diphosphate) is phosphorylated to ATP (adenosine (triphosphate). (This is why the process is called oxidative phosphorylation.)

During electron transport, the participating protein complexes release protons from the matrix to the intermembrane space. This creates a concentration gradient of protons that another protein complex, Complex V, ATP synthase, uses to power synthesis of the energy carrier molecule ATP.

Although the mitochondrion has its own mtDNA, a vast majority of mitochondrial proteins are synthesized from nuclear genes (the DNA within another cell organelle, the cell nucleus) and transported into the mitochondria. These include, but are not limited to the enzymes required for the citric acid cycle, the proteins involved in DNA replication and transcription, and ribosomal proteins. The protein complexes of the respiratory chain are a mixture of proteins encoded by mitochondrial genes and proteins encoded by nuclear genes. Proteins in both the outer and inner mitochondrial membranes help transport newly synthesized, unfolded proteins from the cytoplasm into the matrix, where folding ensues.

### Genetic Factors of Mitochondrial Proteins

Both nuclear and mitochondrial genes have been associated with disease by correlation with genetic mutation.

All 13 Of the proteins encoded by the mitochondrial genome: MTND1, MTND2, MTND3, MTND4, MTND4L, MTND5, MTND6, MTCY8, MTCO1, MTCO2, MTCO3, MTATP6 and MTATP8, have mutations associated with disease. These proteins are generally found at mitochondrial inner membranes.

Nuclear genes encoding mitochondrial proteins (most likely found associated with or bound for the mitochondrial outer membrane) whose mutation has been linked to mitochondrial disease include but are not limited to: ARMS2, BCL2, CPT1A, DNM1L GCK, GK, KIF1B, MAOA, PINK1.

Nuclear genes encoding mitochondrial proteins (most likely found associated with or bound for the mitochondrial inter membrane space) whose mutation has been linked to mitochondrial disease include but are not limited to: AK2, DIABLO, GATM, GFER, HTRA2, PANK2 and PPOX.

Nuclear genes encoding mitochondrial proteins (most likely found associated with or bound for the mitochondrial inner membrane) whose mutation has been linked to mitochondrial disease include but are not limited to: ABCB7, ACADVL ADCK3, AGK, ATPSE, C12orf62, COX412, COX6B1, CPT2, CRAT, CYCS, CYP11A1, CYP11B1, CYP11B2, CYP24A1, CYP27A1, CYP27B1, DHODH, DNAJC19, FASTKD2, GPD2, HADHA, HADHB, HCCS, L2HGDH, MMAA, MPV17, NDUFA1, NDUFA2, NDUFA9, NDUFA10, NDUFA11, NDUFA12, NDUFA13, NDUFB3, NDUFB9, NDUFV1, NDUFV2, NDUFS1, NDUFS2, NDUFS3, NDUFS4, NDUFS6, NDUFS7, NDUFS8, OPA1, OPA3, PDSS1, SDHA, SDHB, SDHC, SDHD, SLC25A3, SLC25A4, SLC25A12, SLC25A3, SLC25A15, SLC25A19, SLC25A20, SLC25A22, SLC25A38, SPG7, TIMM8 UCP1, UCP2, UCP3, UQCRB and UQCRQ.

Nuclear genes encoding mitochondrial proteins (most likely found in or bound for the mitochondrial matrix) whose mutation has been linked to mitochondrial disease include but are not limited to: AARS2, ACAD8, ACAD9, ACADM, ACADS, ACADSB, ACAT1, ALAS2, ALDH2, ALDH4A1, ALDH6A, AMT, ATPAF2, AUH, BCAT2, BCKDHA, BCKDHB, BCS1L C8orf38, C10orf2, C12orf65, C20orf7, COA5, COX10, COX15, CPS1, D2HGDH, DARS2, DBT, DECR1, DGUOK, DLD, DLAT, DMGDH, ETFA, ETFB, ETFDH, FOXRED1, FH, GCDH, GCSH, GFM1, GLUD1, HADH, HARS2, HIBCH, HMGCS2, HMGCL, HSD17B10, HSPD1, IDH2, IDH3B, ISCU, IVD, KARS, MCCC1, MCCC2, MCEE, ME2, MRPS16, MRPS22, MTFMT, MTPAP, MUT, NAGS, NDUFAF1, NDUFAF2, NDUFAF3, NDUFAF4, NUBPL, OAT, OGDH, OTC, OXCT1, PC, PCCA, PCCB, PCK2, PDHA1, PDHB, PDHX, PDP1, POLG, POLG2, PYCR1, RARS2, RMRP, SARDH, SARS2, SCO1, SCO2, SDHAF1, SDHAF2, SOD2, SUCLA2, SUCLG1, SURF1, TACO1, TK2, TMEM70, TRMU, TSFM, TTC19, TUFM, UNG, XPNPEP3 and, YARS2.

Nuclear genes encoding mitochondrial proteins (but proteins that are also found in other places in the cell) whose mutation has been linked to mitochondrial disease include but are not limited to: AIFM1, AKAP10, AMACR, APTX, BAX, BOLA3, CYB5R3, ETHE1, FXN, GDAP1, HK1, HLCS, LRPPRC, LRRK2, MFN2, MLYCD, NFU1, PARK2, PARK7, SACS, SPG20 and WWOX.

Nuclear genes encoding mitochondrial proteins (but whose specific localization within the mitochondrion is still to be elucidated) whose mutation has been linked to mitochondrial disease include but are not limited to: GLRXS, HOGA1, MMAB, MMADHC, PDSS2, AFG3L2, COQ2, COQ6, COQ9, GLDC, PNKD, PUS1, REEP1, STAR and TMEM126A.

Functions of these genes and their products are known in the art. A listing of these genes and other information can be found, for example, in NEJM 2012; 366:1132-41, Supplementary Appendix, and is not repeated here.

Mitochondrial genes in general undergo post-translational modification. Accordingly, in the optimization process, embodiments of the present invention may modify any aspect of these genes, including but not limited to: any function associated with the gene, integrity of the gene itself, transcription, and all the factors including expression and post-translational modification and movement within the cell.

Since genetics underlie life functions, genes that do not encode a protein found in mitochondria can also be of extreme importance in optimized cell metabolism. For example, as discussed below the Position of mitochondria within cells is dependent on many proteins. The genes encoding these proteins can also be important in optimization.

Mitochondria are the major source of metabolic energy, and they regulate intracellular calcium levels and sequester apoptotic factors.

Mgm1 and Opa1 are involved in regulating cristae structure. Mgm1 participates in ATPsynthase oligimerization.

Mitochondria are not just cell powerhouses producing ATP. They also are essential for other facets of cell functions required for metabolism. Cell metabolism is accomplished by thousands of enzymes. Many of these enzymes require metals for proper activity and to form coordination complexes. Iron sulfur clusters (ISVC), essential for iron homeostasis in the cell, are a product of mitochondria. Accordingly, mitochondria through this contribution to iron control, are necessary for many oxidation reactions, including, but not limited to: oxidative phosphorylation, pyrimidine/purine metabolism, the tricarboxylic acid cycle, acontinase activity, DNA repair, NTHL1 activity, heme synthesis, ferrochelatase function, ISC synthesis enzymes (NBP35 and CFD 1). Metal containing enzymes, of which iron containing oxidation/reduction enzymes are common, are important for scavenging active oxygens. For example: FtMt is an important nuclear encoded mitochondrial protein that sequesters iron in mitochondria and makes it available when needed. Mdm33 is important for inner membrane fission. Proton pumping is coupled to ATP synthesis through F₁F₀ATP synthase.

Biochemicals

It is appreciated from the above discussion and from the science of molecular biology and biology in general that many biochemicals are essential or beneficial for proper cell metabolism. Depending on the contribution of the biochemical to the cell processes, the biochemical, may serve, for example, as a chemical substrate, a carrier, a structural member, a signal modifying activity of other biochemicals, etc. Biochemicals, including biotherapeutics, may be delivered by any effective method or device, including, but not limited to: injection, oral dosing, nanodelivery, suppository, patches, eye drops, nasal spray, ointment, cream, synthetic gene, conjugated molecule, catheter, timed or controlled release capsule or pill, subdermal implants, diet, etc. Changing location or activity of one may affect utilization of several others. Although not all of these intertwining pathways are mentioned in detail herein, any one or combination of the metabolic biochemicals and/or the biochemical enzymes processing them can be proper targets for optimization.

Targets, including, but not limited to:

### Riboflavin (B₂)

### L-Creatine

### CoQ₁₀

### L-arginine

### L-carnitine

### Vitamin C

### Cyclosporin A

### Manganese

### Magnesium

### Carnosine

### Vitamin E

### Resveratrol

Alpha lipoic acid
Folinic acid

### Dichloraoacetate

### Succinate

Prostaglandins (PG)—specific to the PG and tissue may show positive/negative effect; e.g., PGA, PGA₂, PGB, PGB₂, PGC, PGD, PGD₂, PGE, PGE₁, PGE₂, PGE₃, PGF_{α}, PGF₁α, PGF2α, PGF₃α, PGG, PGH, PGH₂, PGI, PGJ, PGK, and related biomolecules, including, but not limited to: prostacyclins, thromboxanes, prostanoic acid, 2-Arachidonoylglycerol, etc.
NSAIDS—aspirin—COX1 and COX2 inhibitors

### Melatonin

### Cocaine

### Amphetamine

AZT and similar antiviral compounds
Mitophagic or mitophagic inhibitory compounds: including, but not limited to: isoborneol,
piperine, tetramethylpyrazine, and astaxanthin

### Glutathione

β-carotene and other carotenoids
and as further described below, to provide examples of optimization processing, are deliverable to cells and can be used in optimization as discussed in this application.

Some Representative Compounds and their Importance

Riboflavin

Riboflavin (vitamin B2) works with the other B vitamins. It is important for body growth and red blood cell production and helps in releasing energy from carbohydrates.

L-creatine

Creatine is a naturally-occurring amino acid (protein building block) found in meat and fish, and also made by the human body in the liver, kidneys, and pancreas. It is converted into creatine phosphate or phosphocreatine and stored in the muscles, where it is used for energy. During high-intensity, short-duration exercise, such as lifting weights or sprinting, phosphocreatine is converted into ATP.

CoQ₁₀

There are two major factors that lead to deficiency of CoQ₁₀ in humans: reduced biosynthesis, and increased utilization by the body. Biosynthesis is the major source of CoQ₁₀. Biosynthesis requires at least 12 genes, and mutations in many of them are known to cause CoQ deficiency. CoQ₁₀ levels can also be affected by other genetic defects (such as mutations of mitochondrial DNA, ETFDH, APTX, FXN, and BRAF, genes that are not directly related to the CoQ₁₀ biosynthetic process)

Toxicity is not usually observed with high doses of CoQ₁₀. A daily dosage up to 3600 mg was found to be tolerated by healthy as well as unhealthy persons. However, some adverse effects, largely gastrointestinal, are reported with very high intakes.

L-Arginine

Arginine can be made by most mammals. However, normal biosynthetic pathways, produce insufficient amounts of arginine so some must still be consumed through diet. Arginine is the immediate precursor of nitric oxide (NO), urea, ornithine, and agmatine. Arginine is also a necessary precursor for the synthesis of creatine and other cell component biochemicals. The enzyme, arginase, is found in mitochondrial membranes and here contributes to proper function of the urea cycle. The metal, manganese, is also important for mitochondrial activity at least through its participation in arginine metabolism.

L-carnitine

Carnitine is involved in the transport of acyl-coenzyme A across the mitochondrial membrane to be used in mitochondrial f-oxidation.

Vitamin C

Vitamin C reduces the exercise-induced expression of key transcription factors involved in mitochondrial biogenesis. These factors include peroxisome proliferator-activated receptor co-activator 1, nuclear respiratory factor 1, and mitochondrial transcription factor A. Vitamin C also prevented the exercise-induced expression of cytochrome C (a marker of mitochondrial content) and of the antioxidant enzymes superoxide dismutase and glutathione peroxidase. Vitamin C is an antioxidant, that along with resveratrol and alpha-lipoic acid reduces excessive reactive oxygen species production by the mitochondria. Manganese is also important here as vitamin C works with manganese superoxide dismutase.

Cyclosporin A

Cyclosporin A, an immune suppressant, interferes with the mitochondrial permeability transition pore and therefore has been found effective in protecting against oxidative stress in for example, stress inducing ischemia and reperfusion. Cyclosporin A can improve metabolism in some instances by slowing or blocking cell apoptosis.

Manganese

Manganese plays an essential role in the mitochondrial antioxidant: manganese superoxide dismutase. Without adequate manganese, superoxide dismutase activity will be insufficient, and therefore can result in sub-optimal mitochondrial activity and cellular metabolism.

Magnesium

Magnesium is important for proper calcium metabolism and function as a cofactor with many enzymes. Magnesium also appears especially important for mitochondrial biogenesis.

Zinc

Zinc is important in mitochondrial activity, for example, zinc can affect ATP production rates.

Carnosine

Carnosine is a potent scavenger of free radicals

Vitamin E

Vitamin E is a protectant against mitochondrial membrane peroxidation and therefore can be an important factor in maintaining mitochondrial activity and cellular metabolism.

Resveratrol

Resveratrol is a potent antioxidant with apparent involvement in mitochondrial biogenesis. Resveratrol acts through AMPK and SIRT1 and is involved in PGC-1α.

Alpha-Lipoic Acid

Alpha-lipoic acid is associated with rejuvenation and replacement of damaged mitochondria. This renewal becomes more prevalent as mitochondria age.

Folinic acid

Folinic acid is a factor in mitochondrial oxidative stress and has been associated with mitochondrial dysfunction in autism spectrum.

Dichloraoacetate (DCA)

DCA stimulates oxidative phosphorylation by inhibiting pyruvate dehydrogenase kinase. DCA potency in a particular cell or individual metabolic profile. DCA has been investigated as a possible therapy in some cancers.

Succinate

Succinate is an intermediate in the tricarboxylic acid cycle (making ATP), and participates in inflammatory signaling. Succinate dehydrogenasase participates in electron transport as “Complex II”.

Prostaglandins (PG)

Calcium ion controls binding of many PGs to mitochondria thereby modifying many aspects of mitochondrial function. NSAIDS have been associated with decoupling activity in mitochondria.

NSAIDS—aspirin—COX1 and COX2 Inhibitors

NSAIDS are active in controlling mitochondrial Complex I. NSAIDS may also alter mitochondrial membrane permeability by opening the mitochondrial permeability transition pore that allows small molecules up to 1.5 kDa easier passage across the mitochondrial membrane.

Melatonin

Melatonin demonstrates cell protectant activity though slowing apoptosis as it controls activity of aged or oxidatively stressed mitochondria involvement in leading the cell down the apoptotic pathway.

Cocaine

The anesthetic, cocaine, has been observed as modifying Complex I activity in mitochondria.

Amphetamine

The stimulant class of amphetamines, are inhibitors or normal mitochondrial metabolism and appear to increase oxidative stress.

AZT and similar antiviral compounds

AZT is mitochondrially active by increasing active oxygen generation, interacting with respiratory chain enzymes and damaging mtDNA. Thus optimization of mitochondrial function is a special need when drugs of this type are used.

Mitophagic or mitophagic inhibitory compounds: including, but not limited to: isoborneol, piperine, tetramethylpyrazine, and astaxanthin

Mitophagy is important for recycling of mitochondria and controlling position and number of mitochondria. Either slowing or accelerating mitophagy may be important for optimizing metabolism in a particular cell or individual.

Glutathione

Increased glutathione is known to protect mitochondria and the cell against damaging effects of the oxidative moieties produced in mitochondria such as: superoxide anion radical O₂⁻, hydrogen peroxide, H₂O₂, and the extremely reactive hydroxyl radical HO. Increasing intracellular glutathione content is possible by several methods including, but not limited to: supplying precursors for glutathione synthesis, e.g., N-acetylcysteine; increasing CoA, for example, by supplying its precursor pantothenic acid; making curcumin (a spice) available to the cell; and the analgesic drug flupirtine. Since glutathione is seen to increase throughout the cell, the antioxidant protection is not limited to the mitochondria.

β-carotene

β-carotene, lycopene, lutein, astaxanthin and zeaxanthin are popular carotenoids. These biochemicals demonstrate antioxidation properties. These tend to be lipophilic and thus often are found partitioned in membranes. So at high concentrations they may disorganize normal membrane structure. Cautious treatment with one or more carotenoids can protect membranes against oxidative stress by inhibiting mitochondrial active oxygen production. At least in some cells carotenoids increase mitochondrial function while limiting active oxygen generation. Cell survival can be improved. If the cell whose health is improved is, for example, a cancer cell, then sometimes reduced carotenoids may be advantageous. Optimization here and with other modifications will depend on the disease, the individual and the cell and cell function targeted.

### Mitochondrial Structure and Positioning

As more has been learned about mitochondria it is apparent they are dynamic organelles. From the earliest citing of the mitochondrion over a half century ago we now understand that mitochondrial shape and size are highly variable. Shape and size is controlled by fusion and fission processes. We can also observe that mitochondria are actively transported in cells depending on energy needs within the cell. More mitochondria become situated in areas with higher energy needs, including, but not limited to: active growth cones, presynaptic sites and postsynaptic sites. Also, the internal structure of mitochondria can change in response to their physiological state.

Shape.

Length, shape, size and number of mitochondria are controlled by fusion and fission. Fusion will generally result in fewer, larger and more spherical mitochondria. Whereas high fission cells generally have more mitochondria that are smaller and rod shaped.

Outer shape is not the sole shape criterion. Mitochondria also have internal structure (e.g., shape of cristae). The cristae are regions of the inner membrane more distant (internal) from the outer membrane. Cristae are formed by internal folding of the inner membrane. The different portions of the inner membrane have different functions. For example, cristae are richer in oxidative phosphorylation machinery are more prevalent in cristae while transport facilitators are more prevalent in the inner membrane regions apposite the outer membrane. Not surprisingly, the density and length of cristae are controlled according to the cell's needs and the needs of specific location within the cell.

Location.

One factor controlling mitochondrial movement is its membrane potential. Higher potential favors movement away from the cell nucleus or main cell body towards the periphery. Lower potential (possibly damaged mitochondria) migrate towards the cell center (possibly for destruction).

Signals such as a nerve growth factor (NGF) gradient act to recruit mitochondria to higher concentrations of NGF. These types of factors may be used as a piece of an optimization process to recruit mitochondria to targeted sites. Blocking nerve growth factor activity has been associated with bone cell necrosis.

Within the cell, mitochondria use the cytoskeleton as a guide to destination and for transportation.

Mitochondria are now known to migrate throughout cells, to fuse, and to divide as mitochondrial activity is regulated according to the cell's needs. The dynamic mitochondrial processes enable mitochondrial recruitment on demand to the changing more active subcellular compartments.

Fusion processes as cells converge upon one another and merge facilitates content exchange between mitochondria and is a component of mitochondrial shape control. Stem cells which can fuse with endogenous cells may be involved in rescuing cells with damaged or otherwise dysfunctional mitochondria. Microinjection is an available means of introducing mitochondria to specific cells.

Movement is also important for mitochondrial communication with the cytosol and mitochondrial quality control. For example, when the transmembrane potential of a mitochondrion is diminished the mitochondrion is transported towards the nucleus where mitophagy optimally occurs. A depolarized mitochondrion is an inefficient or damaged mitochondrion; transport to the nuclear region is thus part of a cell's culling process allowing mitochondrial replacement. With these activities mitochondria readily adapt to changes in cellular requirements and therefore can respond to physiological or environmental imperatives.

When mitochondrial dynamics becomes disrupted, cellular metabolism is changed. Accordingly, optimization of cellular metabolism may involve modifying mitochondrial dynamics perhaps by slowing or accelerating translocation of mitochondria in greater proximity to the cell center.

TABLE 1
Proteins involved in Mitochondrial Morphology, Distribution, and Retention in Budding Yeast

| Gene | Protein | Localization | Mutant phenotype |
|---|---|---|---|
| Actin | Mitochondrial morphology | Mitochondrial movement | Mitochondrial |
| cytoskeleton | | | inheritance |
| ACT1 | Actin | Actin patches and actin cables | na |
| ARP2, ARC35, | Arp2/3 complex subunits | Actin patch and mitochondria | Delocalized patches |
| ARC40, ARC15 | | | |
| BNI1, BNR1 | Formins, stimulate actin | Actin cable assembly sites | Loss of actin cables |
| | polymerization | | |
| CCT4, CCT6 | CCT complex required for | Cytosol | Disorganized |
| | actin folding | | |
| COF1 | Cofilin | Actin patches | Defect in actin |
| | | | dynamics |
| IQG1 | Homolog of mammalian | Contractile ring | Disorganized |
| | IQGAP's | | |
| JSN1 | Pumilio family protein, binds | Punctate structures on | Normal |
| | to Arp2/3 complex | mitochondrial surface | |
| MDM1 | Intermediate filament-like | Cytosol | Normal |
| | protein | | |
| MDM2 | Fatty acid desaturase | nd | Normal |
| MDM10 | Mitochore subunit | Mitochondrial outer membrane; | Normal |
| | | punctate structures near mtDNA | |
| | | nucleoids | |
| MDM12 | Mitochore subunit | Mitochondrial outer membrane; | Normal |
| | | punctate structures near mtDNA | |
| | | nucleoids | |
| MDM20 | Subunit of a protein acetylase, | Cytosol | Short actin cables |
| | regulates tropomyosin activity | | |
| MDM31 | Genetic interactions with | Mitochondrial inner membrane | Normal |
| | mitochore | | |
| MDM32 | Genetic interactions with | Mitochondrial inner membrane | Normal |
| | mitochore | | |
| MLC1 | Essential light chain for | Bud tip and contractile ring | Normal |
| | Myo2p and Myo1p | | |
| MMM2 | Mitochondrial outer | Punctate structures near mtDNA | nd |
| | membrane protein | nucleoids | |
| MMR1 | Binds to Myo2p | Bud tip | Normal |
| TPM1, TPM2 | Tropomyosins | Actin cables | Loss of actin cables |
| YPT11 | Rab-like protein, binds to | Bud tip | Normal |
| | Myo2p | | |
| MYO2 | Type V myosin, motor for | Bud tip | Delocalized actin patches |
| | transport along actin cables | | |
| PYF1 | Profilin | Actin patches | Defects in actin dynamics |
| TPM1, TPM2 | Tropomyosins | Actin cables | Loss of actin cables |
| YPT11 | Rab-like protein, binds to | Bud tip | Normal |
| | Myo2p | | |
| Gene | Mitochondrial Morphology | Mitochondrial | Mitochondrial |
| | | Movement | Inheritance |
| Actin | na | na | na |
| cytoskeleton | | | |
| ACT1 | Fragmented, aggregated | Anterograde: none | Decreased, loss of |
| | | retrograde: none | mtDNA |
| ARP2, ARC35, | Tubular, fragmented, | Anterograde: none | |
| ARC40, | clumped | retrograde: normal | |
| ARC15 | | | |
| BNI1, BNR1 | Fragmented, spherical | Anterograde: none | nd |
| | | retrograde: none | |
| CCT4, CCT6 | Fragmented, aggregated | nda | nd |
| | tubules | | |
| COF1 | Fragmented | nd | nd |
| IQG1 | Fragmented | nd | nd |
| JSN1 | Fragmented, aggregated | Anterograde: none | nd |
| | | retrograde: normal | |
| MDM1 | Fragmented, aggregated | nd | Decreased |
| MDM2 | Fragmented, aggregated | nd | Decreased |
| MDM10 | Spherical | Anterograde: none | Decreased, Loss of |
| | | Retrograde: none | mtDNA |
| MDM12 | Spherical | Anterograde: none | Decreased, Loss of |
| | | Retrograde: none | mtDNA |
| MDM20 | Normal | nd | Decreased |
| MDM31 | Spherical, ring like | none | Decreased, loss of |
| | | | mtDNA |
| MDM32 | Spherical, ring like | none | Decreased, Loss of |
| | | | mtDNA |
| MLC1 | Tubular, fragmented | nd | nd |
| MMM1 | Spherical | Anterograde: none | Decreased, loss of |
| | | retrograde: none | mtDNA |
| MMM2 | Distorted/spherical | nd | nd |
| MMR1 | Normal | Normal | Delayed inheritance, |
| MYO2 | Collapsed, tubular | Normal | Delayed inheritance, |
| | | | defects in retention at |
| | | | the poles |
| PYF1 | Fragmented, ring-like | nd | nd |
| TPM1, TPM2 | Fragmented | nd | Decreased; loss of |
| | | | mtDNA |
| YPT11 | Normal | Normal | Delayed inheritance, |
| | | | defects in retention at |
| | | | the poles |

Table 1 from “Interactions of mitochondria with the actin cytoskeleton”, Istvan R. Boldogh and Liza A. Pon. [http://www.sciencedirect.com/science/article/pii/S0167488906000486]

Table 1 lists proteins involved with morphology, distribution and/or retention in yeast cells. Given similarities shared by eukaryotic cells, especially with respect to the mitochondria, these genes, any function associated with the gene, transcription, and all the factors including expression and post-translational modification and movement within the cell, can be targets for optimization.

Mitochondrial movement is controlled by the cell to maintain metabolism. It is generally accepted that kinesin and cytoplasmic dynein regulate the transport of anterograde and retrograde mitochondria. Kinesin-1 and cytoplasmic dynein are tightly coupled in the mammalian prion protein vesicle motor complex. It is believed that kinesin and cytoplasmic dynein are tightly coupled in the mitochondrial-motor protein complex. LIS1 interacts with both KIF5b and DIC so is associated with the connection of kinesin and cytoplasmic dynein. All appear important for mitochondrial movement and positioning at least in some cells.

Fusion motility is powered by GTP (guanosine triphosphate) through Fzo1 and Mgm1 GTPases in yeasts and MFN1 and MFN2 in mammals. Mgn1 is a dynein related protein essential for fusion of the mitochondrial inner membrane. OPA1 and its conjugating partner are active in fusing mitochondrial outer membranes. An electrical potential across the mitochondrion is necessary for fusion. Thus modifying the potential, e.g., by decreasing metabolism or introducing an ionophore can slow or stop fusion.

### Nde11, NudCL LIS1 and Dynein are Important for Mitochondrial Migration in Neurotissue.

The distribution of desmin within striated muscle suggests that it could function as a linkage between mitochondria and myofibrils. In addition to these structural defects, abnormalities in mitochondrial appearance were also observed. Considering the potential association of IFs with mitochondria described above and the suggestions for their possible involvement in mitochondria function. In addition to regulating mitochondrial positioning, it has been postulated that interactions of the cytoskeleton with mitochondria may modulate mitochondrial function. Mitochondrial function could be influenced by changes in mitochondrial shape, by stretching and by contraction of the mitochondrial membrane, which could be directed via the cytoskeleton. Additionally, mitochondrial function could also depend on defined interactions of outer mitochondrial membrane proteins with specific cytoskeletal proteins or cytoskeleton-associated proteins. Specifically, it has been postulated that the cytoskeleton somehow plays a role in the affinity of mitochondria for ADP.

Mutations in IF proteins that cause the disruption of IF networks also alter the morphology, distribution, and functions of mitochondria. For example, mutations in desmin IFs cause changes in the distribution and function of mitochondria in skeletal muscles and heart and a mutation in the neurofilament light chain that causes Charcot-Marie-Tooth disease results in the clustering of mitochondria in the cell bodies of neurons. In keratinocytes of patients with epidermolysis bullosa simplex, caused by mutations in keratins 5 and 14, there is an abnormal distribution of mitochondria and in hepatocytes expressing mutant keratins 8 and 18 there is enhanced susceptibility to apoptosis due to abnormalities in mitochondria. Morphological and functional changes in mitochondria have also been reported in vimentin-null fibroblasts.

Optimization may thus involve consideration of the number of mitochondria, location of mitochondria, size of mitochondria, size and shape, internal structure of mitochondria in addition to chemical factors that may more specifically modify one or more mitochondrial function.

### Process of Optimization

Optimization of cellular metabolism through optimizing any mitochondrial function is desired for improved medical treatment. Cellular metabolism can be observed by any known method or any method that may become known and is not restricted to the examples discussed herein. However, examples are provided as a means to demonstrate the ubiquity of applications of the present invention and feasibility practicing it.

On a simplistic level mitochondrial function may be improved by what we might deem “appropriate nutrition”. Therapists and individuals have historically been known to supplement the diet with vitamins, nutrient and/or cofactors. To date, a methodologic approach to optimizing metabolism specific to an individual or group has not been practiced.

In many patients more complete optimization will involve sequencing their mtDNA. The entire mitochondrial genome can be sequenced or select genes or regions might be deemed of greatest importance. Any one or more of the mitochondrial genes are candidates for sequencing. Sequencing is known in the art and can be accomplished by any successful methodology. Regions of particular interest including, but not limited to: the D-loop or control region might be sequenced to guide optimization protocols. Simply determining total mtDNA in a cell, tissue or individual may also be a step in optimization.

The mtDNA sequence results may be combined with genetic sequence information from one or more organs or cell types in an individual. Genomic sequence is one level of information that may be used in isolation or in combination with mtDNA sequence information for additional guidance in the optimization process. Even more robust information may be obtained, not just from gene expression profiling. This is very useful when considering specific organs or cell types which by being differentiated cells only express a small subset of the full genome. Obtaining RNA transcription profiles or expression profiles can thus be instrumental in the optimization process. In some circumstance analyzing proteins as discussed below with specific reference to blood and other ex vivo biopsy sources, can provide some genomic profile information by monitoring the end product of genomic expression. Accordingly, genomic information in isolation or more preferably in combination with clinical observation and other assays is understood to be a useful source of information to use in developing an optimization protocol.

Analysis may involve inhibiting certain mitochondrial functions to assess their performance levels. Also on occasion optimizing metabolism may involve mitochondrial inhibition.

Several examples of inhibitors are discussed as examples.

### Electron Transport Chain Inhibitors

ETC inhibitors per se act by binding and blocking a component the electron transport chain. ETC function can also be inhibited by impairing expression or proper localization of one of the component enzymes or carriers. Inhibiting or blocking the ETC prevents electrons from being passed from one carrier to the next and stops oxidation of oxygen and synthesis of ATP. Since binding is involved the inhibitors act specifically to affect a particular carrier or complex. Binding can be temporary (reversible) or permanent (irreversible). Reversible inhibition may be time or concentration dependent. Irreversible inhibition generally results in total stoppage of respiration via the blocked pathway. Competitive inhibition is one form of reversible inhibition. It allows some oxygen consumption (and ATP synthesis) since a “trickle” of electrons can still pass through the blocked site. Although it allows some oxygen consumption, competitive inhibition may prevent maintenance of a chemiosmotic gradient. In this example the addition of ADP would have no effect on respiration. Some combinations of inhibitors might be used to seek alternative entry points to the ETC.

Rotenone

Rotenone is used as an insecticide. It is toxic to wildlife and to humans as well as to insects. It is a competitive inhibitor of electron transport suitable for testing ability to block respiration via the NADH versus succinate pathway.

Antimycin

Antimycin has been used with combinations of substrates including succinate, NADH or glutamate, and the dye TMPD (N,N,N′,N′-tetramethyl-p-phenylenediamine) along with ascorbic acid.

Cyanide

Cyanide is a reversible inhibitor of cytochrome oxidase.

Some mitochondria have cyanide resistant pathways. Cyanide causes uncoupling. So in the presence of TMPD a dramatic increase in oxygen consumption is observable.

Malonate

Malonate is a competitive inhibitor of succinate metabolism.

### Uncoupling Agents

Uncoupling is where the rate of electron transport is no longer be regulated by the chemiosmotic gradient. The condition is differentiated from electron transport inhibition by the fact that in the latter case, bypassing the block can restore the gradient. In uncoupling, the ETC still functions but is ineffective because of dissipation of the chemiosmotic gradient.

2,4-dinotrophenol (DNP)

DNP is a proton ionophore. It binds protons on one side of a membrane, and then being fat-soluble drifts to the opposite side where it loses the protons. The probability of binding is greatest on the side of the membrane with greatest proton concentration, and least on the side with the lesser concentration. This makes it impossible to maintain a proton gradient.

DNP demonstrates other effects in addition to uncoupling. DNP gradually inhibits electron transport itself as it incorporates into mitochondrial membranes. In the 1930s DNP was promoted as an effective diet pill. Uncoupling of electron transport from ATP synthesis allows rapid oxidation of Krebs substrates and promotes mobilization of carbohydrates and fats to maintain normal levels of the Krebs substances. The energy is lost and measurable as heat.

Carbonyl cyanide p-[rifluoromethoxyl]-phenyl-hydrozone (FCCP)

FCCP is an ionophore, completely dissipating the chemiosmotic gradient while leaving the electron transport system uninhibited.

Oligomycin

Oligomycin, blocks ATP synthase by blocking the proton channel. This inhibits oxidative phosphorylation. Oligomycin has no effect on Complex IV respiration, but blocks Complex III respiration completely. It therefore has no direct effect on electron transport or the chemiosmotic gradient.

### Any Mitochondrial Function or Related Function is a Possible Target for Optimization

Cells and mitochondria each and collectively require multiple metabolic functions for their own survival and survival of the organism. In any particular cell or condition, modifying a specific function or activity or a select group of metabolic functions or mitochondrial activities may be selected for optimization, in other cells or conditions, including, for example, cells of a different organ with the same individual or cells of a different individual. Such activities that might be altered associated with the optimization process may include but are not limited to: oxidative phosphorylation, energy versus heat production (efficiency), free radical generation, free radical scavenging, initiation of apoptosis, mtDNA transcription, mitochondrial protein translation, post translational modification, mitochondrial protein import or translocation, nucleotide translocation, ATP translocation, mitochondrial fission, mitochondrial fusion, Ca⁺⁺ compartmentalization or homeostasis, steroid biosynthesis, controlling portions of the urea cycle, fatty acid oxidation, the tricarboxylic acid cycle, pyruvate metabolism, cellular redox balance, synthesis of precursor compounds such as myelin precursors, altering iron metabolism and of course altering oxygen use and any component or activity of the electron transport chain. [Generation of metabolites to regulate cellular epigenetics (NAD⁺) methyl group and numerous additional metabolic processes.] The skilled artisan will recognize that optimization of any one or more of these may not be relevant for every cell type. Depending on the therapy at issue, any of these functions or activities or any of the many functions or activities not specifically mentioned here, but appropriate to the condition or cell involved in the treatment, the skilled artisan will select and optimize relevant functions and/or activities. To optimize treatment for the individual, disease status; the individual's history with the disease; the individual's response to the disease; the individual's genetic background (including methylation and other epigenetic control of polynucleic acids or their histones); the individual's biochemical status for one or more markers, metabolites or substrates; and experience such as data from the disease, the individual or any relevant group or subgroup can be used alone or in combination.

Cellular or mitochondrial morphology; e.g., size, number, location, shape, can be used to assess mitochondrial function. One means helpful in this analysis is FACS (fluorescence activated cell sorting). This technology is several decades old and therefore has seen development of a variety of fluorescent markers to indicate location, size, membrane potentials, including mitochondrial membrane potentials inside a cell. FACS is one technique available to assess deficits in mitochondrial form and/or function. Observing a facet of mitochondrial function that may be improved can be used to then select one or more optimizing strategies. Optionally, selected strategies can be tested in cells using repeated FACS, to refine and to further improve and optimize strategy.

Analysis of an individual or a group or class of individuals for normalization or validation can be directed explicitly at reactions carried out by mitochondria. However, this often may require a bioassay, removal of tissue from an individual for ex vivo analysis. And since the mitochondrion is an essential component of eukaryotic cells, participating in multiple metabolic pathways, mitochondrial status can be evaluated by secondary or tertiary parameters. For example, blood can be used to monitor mitochondrial health and therefore may be used in the present invention as a material for bioassay. Several fractions of blood may be used at the discretion of the practitioner. For example, mitochondria themselves can be found in white blood cells. Fibroblasts, mesenchymal stem cells, cancerous and/or cancer progenitor are examples of some rare but observable cell types that can be found in blood. Any cell found in the blood might be used as a source for nucleic acid to assay or sequence a nuclear or mitochondrial genome or a portion thereof.

The blood also carries other components, fatty acids, proteins, glycoproteins, lipoproteins, carbohydrates (simple and complex), gases (especially oxygen and carbon dioxide), ketones, hormones, metabolites, nitrogen compounds, active oxygen molecules, ions (atomic, polyatomic, organic, etc.), amino acids, plasma proteins (such as albumen that may scavenge [bind] drugs or other molecules), cytokines, platelets, molecules carried from the digestive system or lungs, etc. that may be used to indicate, tissue, cell and mitochondrial status. The invention envisages blood as a robust source of information that might be used in the optimization process. Each component may be assayed in its native or altered form. For example, a modified protein or nucleic acid can be very instructive in determining metabolic status. In many embodiments monitoring representative compounds as those discussed above will be useful in developing and monitoring optimization. In several embodiments cytokines, a generic term for interleukins (including, but not limited to: IL-1a, IL-1b, IL1Rn, IL2, IL-3, IL-4, IL-5, IL-6, IL-7, IL-8, IL-9, IL-10, IL-1, Il-12, IL12a, IL12b, IL-13, IL-14, 11-15, IL-16, IL-17, IL-17a, IL7b, 11-18, IL-19, IL-20, Il-21, IL-22, Il-23, IL23a, IL-24, Il-25, IL-26, Il-27, Il-28, IL-29, Il-30, Il-31, Il-32, Il-33, Il-34, Il-35, Il-36, Il-37, etc.), interferons (including, but not limited to: IFN-α, IFN-b, IFN-g, etc.), colony stimulating factors (including, but not limited to: M-CSF GM-CSF, G-CSF, [aka CSF1, CSF2 and CSF3] etc,), tumor necrosis factors (TNFA, Lymphotoxin (TNFB/LTA-TNFC/LTB), TNFSF4, TNFSF5/CD40LG, TNFSF6, TNFSF7, TNFSF8, TNFSF9, TNFSF10, TNFSF11, TNFSF13, TNFSF13B, EDA, etc.), and growth factors (including, but not limited to: BMP2, BMP4, BMP6, BMP7, CNTF, CNTF, GPI, LIF, MSTN, NODAL, OSM, THPO, VEGFA, etc. [Colony stimulating factors are included in this family by some reporters.]) may be assessed to aid optimization. [Naming convention is in flux so many cytokines have multiple designations. For example, one convention identifies 3 families if interferons by type, Type I, Type II and Type III. Each may have 1 or more subtypes, example, in humans, at least 13 α, 1 β and 1 ω subtype have been characterized. K and a subtypes are also known.]

Many drugs targeting cytokines or cytokine receptors have been developed or are under development. Accordingly, cytokine assays may be especially useful in developing optimization protocols since tools are available to modulate effect. Modulation of the endogenous quantities produced by an individual may be an enhancement tool used in some embodiments. Synthetic compounds antagonizing or agonizing of any assayed substance may also be appropriate tools.

Assaying may one blood component might crudely be used to monitor cellular and/or mitochondrial performance. However, there is no practical reason to eschew analysis of other components provide more directed information to guide optimization. Assaying multiple aspects can indicate performance or changed performance to judge an optimization pathway. For example, threshold levels of one or more blood components may indicate a certain level of activity of one or more metabolic pathway. Beyond simple thresholds ratios of two or more components, by showing relationships, can provide more definitive information. Diurnal or other periodic relations may also guide optimization. Sometimes more complex algorithms getting at multi factor relationships (multiple pathways, serial pathways or parallel pathways, different organs, for example). Computer learning or other forms of artificial intelligence is now becoming a more accepted process to determine most effective analysis criteria.

While blood is a great source for a substantial number of components or factors that can be assayed, the body has other assayable tissues including, but not limited to: cerebral spinal fluid, lymph fluid, saliva, breath, tears, urine, sweat, mucus, gastric and/or intestinal contents, stool, etc. Any one or more of these tissues or components can be used individually or in conjunction with one or more other source to provide data used in optimization.

Analysis may be accomplished using any acceptable means such as categorization, parametric statistics, nonparametric statistics, ratio analysis, simple or complex comparisons, threshold assays, computer learning, etc.

Analysis may be repeated to assess degree of optimization and/or to assist in determining any change or addition to the optimization process. Analysis may also be repeated with any changed condition of the treatment recipient. Several repetitions of analysis and modified optimization process may be conducted in an iterative fashion.

Cellular metabolism or mitochondrial function may be optimized for an individual, even for an individual during a particular season, time of day, sleep-wake cycle, etc. Optimization may be based on data collected from more than one individual. For example, an optimized process may be determined for a select grouping. The skilled artisan will have capability to select an appropriate group, based for example on similarities within a group. If data show insignificant variability pooling is more appropriate.

Groupings may be based on disease or stage of disease. Groupings may be based on familial connections or larger genetic associations. For example, groups may be categorized from associations including, but not limited to: shared ancestry; shared country or region of familial origin; shared blood type (possibly subtypes); A, A1, A2, B, B1, etc.), shared Rh factor (possibly considering each or a combination of Cc, Dd, and Ee), any of the other grouping systems including, but not limited to: ABO, MNS, P, RH, LU, KEL, LE, FY, JK, DI, YT, XG, SC, DO, CO, L, CH, H, XK, GE, CROM, KN, IN, OK, RAPH, JMH, I, GLOB, GIL, RHAg, FORS, LAN, JR, Vel, CD59; HLA; one or more of the 4 main mitochondrial clusters with multiple DNA lineages; one or more of the 7 core mtDNA lineages (U, X, H, V, T, K, J); one or more of the nineteen mtDNA groups (A, B, C, D, F, G, H, I, J, K, L, M, N, U, V, W, and X); shared diet; shared eye color; shared gender; shared body type; similar height; similar weight; similar BMI or other biometric.

Generally, any assay might be used as part of the cell optimization process to assess one or more components of cell metabolism and/or mitochondrial activity. Some common types include but are not limited to: end point assays, kinetic assays, qualitative, semi-qualitative or quantitative assays, functional assays, immunoassays, radio-assays, fluorescent assays, binding assays, enzymatic assays, isotopic assays, mass spectrometry, photo-assays, cell sort assays, spectrophotometry, polymerase chain reaction, laser coupled assays, agglutination assays, transmittance, absorbance, refraction, flow assays, size assays, ion assays, conductivity assays, uptake assays, secretion assays, mass, gel electrophoresis, transport of: DNA, RNA, proteins, or presence or amount of specific sequences, toxicity assays, viability assays, chemiluminescent assays, amino acid assays—amino acid ratio assays, carbohydrate analysis, biomarker assays, etc.

Less specific assays can also be used to select optimization strategy. For example, fairly routine analysis of a biosubstance, e.g., a body fluid (for example: urine, blood, sweat, cerebral-spinal fluid, saliva) for one or more commonly seen components (for example: any of the amino acids, glucose or other monomeric compounds. One or more of the collagens may be observed to assess initial status and/or to monitor progression of the optimization strategy. For example, condition of the skin might be scored to chart effectiveness of treatment since skin is easily accessible and collagen is ubiquitous throughout the body's organs. As an example, collagen VI or a correlated marker might be monitored to assess Alzheimer's disease. Collagen monitoring may also be beneficial in tracking cancer growth and optimized treatment effectiveness. Assaying one or more biosubstance obtained, for example, from natural elimination or biopsy is considered important to many embodiments of the present invention.

This process of producing and properly distributing ATP for proper cell function is complex and therefore is sensitive to changes to the cell's homeostasis. Accordingly, a necessity for cell survival optimal function and energy metabolism (as manifest, for example, in ETC, protein or peptide synthesis, signal transduction, mitochondrial function, proton gradients and activated phosphates) is easily compromised before the therapy or during therapy that produces other desired effects. Accordingly cell survival can be easily compromised; disruption to these processes can disruptively alter anything else, for example, post translational modification. Cellular energy metabolism needs to be optimized before or during therapy to maximize benefit.

As a description emphasizing complexity, the ETC incorporates three of these proton pumps known as complexes I, III and IV. Notably, complexes I and III catalyze reactions very close to equilibrium. Reactions catalyzed by these complexes are easily reversed and therefore especially sensitive to extracellular events.

Complex II can replace complex I, but is not a proton pump and produces less energy than pathways using complex I. When complex II becomes more active, energy metabolism and therefore the cell becomes less efficient.

Optimization therefore can have many possible pathways. One or more of these may be applied for any individual. For example, the mitochondrial genome encodes 37 genes (16, 569 bp): 13 polypeptides, 22 tRNAs and 2 ribosomal RNAs. The polypeptides are constituents of the respiratory-chain complexes: 7 complex I subunits (NADH dehydrogenase), 1 subunit of complex III (ubiquinol: cytochrome c oxidoreductase), 3 subunits of complex IV (cytochrome c oxidase) and 2 subunits of complex V (ATP synthase). The genes for tRNAs are presented as one-letter symbols. Mutations in four of these tRNA genes are associated with diabetes: those for leucine (L), serine (S), lysine (K) and glutamic acid (E) tRNAs. [http://www.nature.com/nature/journal/v414/n6865/fig_tab/414807a_F1.html]. These, since the mitochondria are essential components of eukaryotic cells, interact with the cellular components produced by nuclear genome of the cell (since many pathways in energy production require genes from both).

Exemplary donor and acceptor compounds in the pathway include the coenzymes nicotinamide adenine dinucleotide (NAD⁺) and flavin adenine dinucleotide (FAD), yielding NADH and FADH₂. Then in the pathway, subsequent oxidation of these hydrogen acceptors leads to the production of ATP.

Since NADH is a component of the ETC, ETC and the mitochondrion are involved in other groups of pathways, for example reduction of disulfides. One such disulfide system is the glutathione system, a system essential for many transport functions within the cell and therefore healing and repair.

Even compounds such as fatty acids by participation in the citric acid cycle affect and/or are affected by any alteration from optimal mitochondrial function. So obesity or even localized fat deposition would be candidates for improvement through optimization of mitochondrial function.

To further highlight complexity of the energy system the following examples of molecules involved in energy metabolism are mentioned: carbohydrates, fats, proteins, acetyl-CoA, CoA-SH, cis-Aconitate, nicotinamide adenine dinucleotide (NAD+), reduced NAD⁺ (NADH), flavin adenine dinucleotide (FAD), FADH2, α-ketoglutarate, guanosine diphosphate (GDP), inorganic phosphate (Pi), guanosine triphosphate (GTP), adenosine diphosphate (ADP), adenosine triphosphate (ATP), hydronium and hydride ions, ubiquinone, and the reduced form ubiquinol, succinate, fumarate, Cytochrome c, isocitrate, oxlosuccinate, succinyl-CoA, L-malate and citrate. At a first level, every treatment altering any concentration, location, availability or enzymes that can use these substances as substrate would alter the energy metabolism set by the cell. In general we should presume the metabolic balance set by the cell (in the absence of treatment) was optimized for at least one function. Restoring proper balance therefore should improve the treatment process.

On the downside of cell regulatory activities, in the past half century or so a class of molecules called reactive oxygen species (ROS) has been implicated in multiple disease etiologies. These are volatile oxygen substances that can initiate, for example, peroxidation chain reactions and may damage DNA as well as other cell components. Common diseases such as cardiovascular disease and many cancers are suspected as having ROS component in their development.

Mitochondrial function, because of its propensity to oxidize substances (chiefly involving oxygen) is therefore implicated in many disease states. Not surprisingly, many treatments for common disease will compromise mitochondrial function. Restoration of better health through optimizing energy metabolism should ideally become an important component of treatment.

In addition to merely optimizing mitochondrial function measured by optimizing the energy output, mitochondrial function may be optimized to treat or prevent some common disease. As mentioned above optimizing mitochondrial function to benefit proper glutathione levels can be considered important both for near term health and prevention or management of future disease.

Antioxidants, such as vitamins and red wines have been used generically, but generally not for specific effect to promote mitochondrial related health. Optimization of energy metabolism involves more than simply adding items to one's diet. Michael Ristow, in a 2009 study, found indeed that antioxidant supplementation (He used vitamins C and E.) had no positive effect. In fact, Ristow's studies were interpreted to conclude that antioxidant supplement left one weaker. So simply adding a molecule that counters an undesired molecule involved in mitochondrial metabolism is definitely not an obvious solution for ameliorating disease treatment or progression.

Enzymes, the catalysts for biologic activity, are important for optimized metabolism. Several of these enzymes require a metal to complete their structure. For example, superoxide dismutases (SODs) essential to detoxify active oxygens (like superoxide), contain either zinc (Zn²⁺) and copper (Cu²⁺) or manganese (Mn²⁺) as in the mitochondrial form. These SODs convert superoxide to peroxide and thereby minimizes production of hydroxyl radical, the most potent of the oxygen free radicals. But the peroxides produced by SOD are also toxic. Peroxidase is the enzyme that detoxifies peroxides. The best known mammalian peroxidase is glutathione peroxidase. This enzyme contains a modified amino acid selenocysteine in its reactive center.

This is perhaps understandable using, for example, Nrf2 as an exemplary intracellular regulator protein. Nrf2 activity is implicated in regulating a gross or more of gene in the cell. Optimization of mitochondrial function may affect Nrf2 activity on concomitantly, optimization of mitochondrial function may be addressed through controlling Nrf2.

In concert with the above discussion, we need to remember that the human organism, including the mitochondria that reside in its cells, have evolved over eons. It is only recently that humans have used medicinal sciences to target invading organisms, dysfunctioning organs or cells, messaging pathways dictating cell activity, or cells' internal components and functions. While often we will have evolved to improve or optimize natural stresses, these newly manufactured stresses will not be managed by systems that through trial and error (evolution) have been optimized to a degree to maximize survival of the species. When a disease affects an organism or with good intentions we presume to modify one part of the cell's activities, because of the interrelatedness of the multiple pathways within a cell, we likely will observe secondary and tertiary or more abstract effects if we look for them. Investigating whether such an important component of the cell, such as the mitochondrion, can have its function improved and taking action to improve function can be expected to show great benefits to the individual.

Mitochondrial function is thus extremely important and changeable. Any mitochondrial gene or any mitochondrial protein gene, their control mechanisms and their products or metabolites should therefore be considered as possible targets in the optimization processes. For example, Slowing MFN1, MFN2 or OPA1 can seriously reduce respiratory capacity. Combination of multiple modifying schemes sometimes can be quite advantageous. For example, generic components, such as lipids (including glycolipids, phospholipids, etc.), substrates, and possibly indicator substances might be introduced while also increasing mitochondrial fusion. The fusion aids in more widespread distribution and delivery. When movement is the goal, increasing fission can make the mitochondria more mobile and enable delivery to cell periphery. Fission is also a facilitator of apoptosis. Accordingly, increasing fission events can aid treatments where apoptosis is desired and decreasing fission can spare cell death.

Energy Related Disease

This list of diseases related to cell metabolism is provided as evidence of the applicability of the present invention. Treatment of these and other diseases can benefit from the optimization processes embodied in this invention.

Nuclear Mitochondrial Disorders.

Cardiomyopathy and encephalopathy with complex I deficiency—mutations in NDFUS2

Optic atrophy and ataxia with complex II deficiency—mutations in SDHA

Hypokalaemia and lactic acidosis with complex III deficiency—mutations in UQCRB

Mutations involving assembly factors of the mitochondrial respiratory chain

Leigh syndrome-mutations in SURF I and LRPPRC

Hepatopathy and ketoacidosis—mutations in SCO1

Cardiomyopathy and encephalopathy—mutations in SCO2

Leukodystrophy and renal tubulopathy—mutations in COX10

Hypertrophic cardiomyopathy—mutations in COX15

Encephalopathy, liver failure, and renal tubulopathy with complex III deficiency—mutations in BCS1L

Encephalopathy with complex V deficiency—mutations in ATP12

Nuclear genetic disorders of intra-mitochondrial protein synthesis

Leigh syndrome, liver failure, and lactic acidosis—mutations in EFG 1

Lactic acidosis, developmental failure, and dysmorphism—mutations in MRPS16

Myopathy and sideroblastic anaemia—mutations in PUS1

Leukodystrophy and polymicrogyria—mutations in EFTu

Encephalomyopathy and hypertrophic cardiomyopathy—mutations in EFTs

Oedema, hypotonia, cardiomyopathy, and tubulopathy—mutations in MRPS22

Hypotonia, renal tubulopathy, and lactic acidosis—mutations in RRM2B

Nuclear genetic disorders of mitochondrial protein import

Mohr-Tranebjaerg syndrome or deafness-dystonia-optic neuronopathy (DDON) syndrome—mutations in TIMM8A (DDP)

Early-onset dilated cardiomyopathy with ataxia (DCMA) or 3-methylglutaconic aciduria, type V-mutations in DNAJC19

Nuclear Genetic Disorders of Mitochondrial DNA Maintenance

Chronic progressive external ophthalmoplegia—mutations in POLG, POLG2, PEO1, SLC25A4, RRM2B, and OPA1)

Mitochondrial neurogastrointestinal encephalomyopathy—mutations in TYMP

Alpers syndrome-mutations in POLG and MPV17

Infantile myopathy and spinal muscular atrophy—mutations in TK2

Encephalomyopathy and liver failure—mutations in DGUOK

Hypotonia, movement disorder and/or Leigh syndrome with methylmalonic aciduria—mutations in SUCLA2 and SUCLG1

Optic atrophy, deafness, chronic progressive external ophthalmoplegia, myopathy, ataxia, and peripheral neuropathy—mutations in OPA

Miscellaneous

Co-enzyme Q10 deficiency—mutations in PDSS2, APTX, COQ2, and ETFDH

Barth syndrome—mutations in TAZ

Cardiomyopathy and lactic acidosis associated with mitochondrial phosphate carrier deficiency—mutations in SLC25A3ncy—mutations in SLC25A3

Alpers syndrome: epilepsy, cortical blindness, micronodular hepatic cirrhosis, episodic psychomotor regression; Barth syndrome: cardiomyopathy, hypotonia, weakness, and neutropenia.

Nuclear mitochondrial disorders represent an important group of human diseases. They often pose significant diagnostic challenges related to their genetic and phenotypic heterogeneity, but they are increasingly being recognized, helped by greater clinical awareness and easier access to molecular genetic testing. A common feature shared by all these disorders is impaired mtDNA maintenance, which can lead to a reduction in mtDNA copy number, the accumulation of high levels of somatic mtDNA mutations, or both. The identification of these quantitative and qualitative mtDNA abnormalities in diagnostic specimens is therefore a key finding, suggesting an underlying nuclear defect, and helping to direct appropriate molecular investigations. MtDNA depletion is the pathological hallmark of several early-onset mitochondrial syndromes, and the clinical prognosis is often poor, due to the marked bioenergetic crisis caused by such a gross reduction in mtDNA copy number (Spinazzola et al., 2009). Interestingly, the observed mtDNA depletion can be highly tissue-specific, which partly explains the variability in disease presentation and severity.

A mosaic pattern of cytochrome c oxidase (COX) deficient fibers is frequently observed in muscle biopsies of patients with both primary mtDNA and nuclear mitochondrial disorders, with some of these fibers exhibiting abnormal accumulation of mitochondria in the subsarcolemmal space, giving the classical appearance of “ragged-red fibers” (RRFs).

Accordingly, the present invention provides an improved method of medical therapy through administering a medicament that is specifically to: the affliction, disease, person, group to which the person belongs, personal activities, and/or therapies or nutrition for the person, selected to balance, restore, optimize and/or enhance the person's cellular metabolism that is deficient, compromised or otherwise determined to be sub-optimal. The function to be improved may be in response to or may be due to a variety of underlying causes that result from one or more events selected from the group consisting of i) a detectable deficit in cellular metabolism, ii) a condition that benefits from therapeutic intervention, and iii) the therapeutic intervention.

Of many possibilities for practicing the invention one example involves improving or restoring cellular metabolism by altering mitochondrial location in a cell. Mitochondrial location may be manipulated or guided by one or more practices including, but not limited to: by controlling cytoskeleton interaction with a mitochondrion, by controlling actin interaction with a mitochondrion, by controlling microtubule interaction with a mitochondrion, by controlling kif5b activity, by controlling Ca⁺⁺ activity, by controlling a cell's cytoskeleton interaction with a mitochondrion including interaction that may comprise a dynein interaction, by controlling a potassium gradient within a cell, by controlling the permeability of a mitochondrion to one or more ions, by controlling vimentin activity, by controlling plectin activity, by changing the location of an interfibrillary mitochondrion, and by changing location of a sub-sarcolemmal mitochondrion, etc.

In several embodiments the administered medicament results in mitochondrial function enhancement or optimization that may be observed in a cell, an organ, a confirming biopsy, an individual, or a group or subgroup of exemplary, corresponding, or related individuals. Defining parameters of the group or subgroup can be any relevant categorization including, but not limited to: age, gender size, diet, genetic history gene analysis, fitness and treatment history.

According to the present invention the emphasis is on an individual and the circumstances unique to that individual. While the present invention acknowledges and strongly supports obtaining data from multiple sources, for example, individuals from different backgrounds and/or disease status in part to help differentiate simple time correlation from genuine and true optimization results, individuals are not identical. The specific circumstance peculiar to each individual must be considered. Accordingly, broad averages are not emphasized in practicing the present invention thus deleterious side effects that are possible in a measurable percentage of a general population will not on that basis disqualify possible use in optimization. For example, a protein inactivated by phosphorylation may be observed in an individual. Optimization in that individual may comprise blocking or inhibiting production of that protein, thus sparing the individual (actually cells of that individual) the chemical and energetic expense of manufacturing the protein and the expense of maintaining the phosphorylation pathway for that protein. The skilled artisan will readily recognize that it is unlikely that the protein at issue would have been evolutionarily retained unless it had a survival benefit. It would therefore be expected that in other individuals or other cells expression of that protein would be beneficial. This example is presented to illustrate that optimization in one instance might involve a treatment opposite that used in another instance.

A medicament that is beneficial in improving or optimizing mitochondrial performance in one individual might in fact be a medicament detracting from performance in another. In an extreme instance a toxic even fatally toxic medicament dose in one individual may show promising enhanced results in another. Caution is thus required in practicing optimization protocols. Assessment of an individual's circumstance with particular attention to differences from general averages (for example an individual may present with one or more SNPs or may have been exposed to an environment stimulating a suboptimal or deleterious corrective reaction) particular to that individual will be instrumental in guiding optimization plans.

The particular circumstance observed may relate to a finding including, but not limited to those where an individual: may be present with a particular disease, may present with a particular genomic sequence or mtDNA sequence, may present with a particular pairing or association of plural noted sequences, may present with presence of a particular protein or modified protein, may present with absence or dearth of a protein, may present with enhanced activity of a metabolic pathway or a part of a metabolic pathway, may present with inability to metabolize a chemical (e.g., a nutrient such as phenylalanine toxic to some but a protein building block in the majority of individuals), may present with a history associated with a disease or disease pattern, may present with an exposure profile that may be unique to the individual or a small group of individuals (think Chernobyl or other workplace or environmental exposure to a physical or chemical event), may present with a familial profile, may present with a detectable imbalance of any of the chemicals common in the body, may present with a misfolded protein (e.g., mad cow protein disease, rare in the general population on earth but common in certain regions or persons with particular genetic characteristics [for example a gene encoding for methionine at position 129] exacerbated by a second particularity at position 178—Met 129 is not generally sufficient to cause mad cow disease, but requires exposure to a mad cow protein; although met-129 is not serious in isolation, either the mad cow prion which elicits mad cow in that individual or the 178 mutation in concert with met-129 leads to fatal familial insomnia), etc.

It is essential that the individual be assessed appropriately in practicing optimization procedures. Since optimization will vary with individual need and if effective will change circumstance of the individual as time progresses, in iterative optimization process is preferred wherein after an initial assessment and enhancement exercise a subsequent assessment is applied to elucidate potential new optimization protocols that might be advantageous to administer.

Especially when a substance that might be or might become toxic might be employed in the enhancement plan, the practitioner is advised to optimization progress to avoid a toxic (sub-optimal) outcome.

It must be understood that a procedure used for optimization in one individual might be severely deleterious in another. For example, clotting factors import to survival in hemophiliacs could be fatal if administered to an individual with a history of deep vein thrombosis or other clotting abnormality. The present invention considers this real possibility, that a substance's toxicity may be quite dependent on individual or timing of administration, in developing many of its embodiments.

In specific embodiments where the medicament administered directly or indirectly results in improved mitochondrial function enhancement or optimization the enhancement or optimization may be confirmed by any of a variety of acceptable methods including, but not limited to: observation in a cell, observation in an organ, observation in an individual, a group or subgroup of individuals, observation by a performance assay, observation by a metabolic assay, observation by one or more clinical criteria, observation using light, sound, heat or particle emission, etc.

Optimization may focus on cellular energy metabolism and/or mitochondrial energy metabolism or may consider the whole organism. Depending on specific circumstance, optimization may involve destruction of mitochondria, either enhanced or diminished destruction, but may also involve altering selective destruction of mitochondria, for example, through autophagy or mitophagy. In some circumstances, optimization may require preservation of damaged mitochondria to preserve needed metabolic function. Contrarily, in other circumstances removing or inactivating damaged mitochondria may enhance outcomes. When the entire organism is considered, elimination of select healthy mitochondria may prove optimizing. Generally the balance of cell types within an organism includes essentially zero malignant or cancerous cells.

Non-malignant growths may also be undesired. One means of decreasing the number, optimally to the point of elimination of the undesired cell type is to turn off the cell's metabolism. Treating mitochondria in a fashion to permeabilize the mitochondrial membrane will cause membrane depolarization. When this is minor, mitochondria will be translocation within the cell towards the nuclear region for destruction. Destroying significant amounts of mitochondria will make these cells less healthy and thus less able to fend off immune attacks or possibly radiation and/or chemotherapeutic attacks. At a higher level of damage to mitochondria, for example, severe permeabilization of the mitochondrial membranes the mitochondria will be unable to support the cell's metabolic needs and even in the absence of extracellular attack can result in death of the cell.

Rather than controlling destruction, an improved treatment method may comprise modulating mitochondrial biogenesis or synthesis, mitochondrial fusion or splitting, mitochondrial location, the number of genomes within one or more classes of mitochondria. Thus the heteroplasmy ratio and/or integrity of mitochondrial genomes comes into play, the beneficial manipulation of which is included in several embodiments of the present invention.

Mitochondrial function enhancement or optimization may be accomplished by several processes including, but not limited to: changing a mtDNA mutation threshold for initiation of mitochondrial destruction (autophagy or mitophagy); the rate of mtDNA mutation, altering iron metabolism in the cell's iron sulfur clusters; altering oxidative phosphorylation through controlling iron metabolism by a mitochondrion; altering pyrimidine/purine metabolism through controlling iron metabolism by a mitochondrion; altering the tricarboxylic cycle through controlling iron metabolism by a mitochondrion; altering heme synthesis through controlling iron metabolism by a mitochondrion; altering the availability to a mitochondrion of a substance selected from the group consisting of Riboflavin (B₂), L-Creatine, CoQ₁₀, L-arginine, L-carnitine, vitamin C, cyclosporin A, manganese, magnesium, carnosine, vitamin E, resveratrol, α-lipoic acid, folinic acid, dichloraoacetate, ssuccinate, prostaglandins (PG) prostacyclins, thromboxanes, prostanoic acid, 2-arachidonoylglycerol, NSAIDS, melatonin, cocaine, amphetamine, AZT, mitophagic controlling compounds, glutathione, β-carotene and other carotenoids, etc.

Prostaglandins PGs) may be instrumental in the optimization process. Many are possible to use in various embodiments of the present invention. Useful PGs include, but are not limited to: PGA, PGA₂, PGB, PGB₂, PGC, PGD, PGD₂, PGE, PGE₁, PGE₂, PGE₃, PGF_{α}, PGF₁α, PGF₂α, PGF₃α, PGG, PGH, PGH₂, PGI, PGJ, PGK, etc.

Non-Steroidal Anti-inflammatory Drugs are known in the art and can find new use in accordance with the present invention. Accordingly, PGs including, but not limited to: Aspirin (acetylsalicylic acid), celecoxib (Celebrex), dexdetoprofen (Keral), diclofenac (Voltaren, Cataflam, Voltaren-XR), diflunisal (Dolobid), etodolac (Lodine, Lodine XL), etoricoxib (Algix), fenoprofen (Fenopron, Nalfron), firocoxib (Equioxx, Previcox), flurbiprofen (Urbifen, Ansaid, Flurwood, Froben), ibuprofen (Advil, Brufen, Motrin, Nurofen, Medipren, Nuprin), indomethacin (Indocin, Indocin SR, Indocin IV), etoprofen (Actron, Orudis, Oruvail, Ketoflam), ketorolac (Toradol, Sprix, Toradol IV/IM, Toradol IM), licofelone, lornoxicam (Xefo), loxoprofen (Loxonin, Loxomac, Oxeno), lumiracoxib (Prexige), meclofenamic acid (Meclomen), mefenamic acid (Ponstel), meloxicam (Movalis, Melox, Recoxa, Mobic), nabumetone (Relafen), naproxen (Aleve, Anaprox, Midol Extended Relief, Naprosyn, Naprelan), nimesulide (Sulide, Nimalox, Mesulid), oxaporozin (Daypro, Dayrun, uraprox), parecoxib (Dynastat), piroxicam (Feldene), rofecoxib (Vioxx, Ceoxx, Ceeoxx), salsalate (Mono-Gesic, Salflex, Disalcid, Salsitab), sulindac (Clinoril), tenoxicam (Mobiflex), tolfenamic acid (Clotam Rapid, Tufnil), valdecoxib (Bextra), etc. may be included in the practice of this invention.

In embodiments where mitophagy or autophagy is controlled the practitioner has available several choice compounds including, but not limited to: isoborneol, piperine, tetramethylpyrazine, and astaxanthin.

Embodiments of the present invention may include multiple components, e.g, administering an antioxidant to enhance results. The antioxidant may result in mitochondrial function enhancement or optimization and may include situations wherein a triphenylphosphonium cation facilitates delivery of the antioxidant.

The improved method may be validated by observation (collecting data from a subgroup of individuals that may be selected from a group of subgroupings including, but not limited to: individuals having shared ancestry; individuals having shared country, individuals sharing a region of familial origin; individuals having a specific blood type; individuals sharing a Rh factor, individuals sharing anyone or any combination of the grouping systems ABO, MNS, P, RH, LU, KEL, LE, FY, JK, DI, YT, XG, SC, DO, CO, L, CH, H, XK, GE, CROM, KN, IN, OK, RAPH, JMH, I, GLOB, GIL, RHAg, FORS, LAN, JR, Vel and CD59; individuals sharing HLA typing; individuals carrying one of the 4 main mitochondrial clusters; individuals having any one of the 7 core mtDNA lineages; individuals having one of the nineteen mtDNA groups; individuals sharing a diet; individuals with the same eye color; individuals sharing a gender; individuals sharing a body type; individuals of similar height; individuals of similar weight; individuals with similar BMI and individuals sharing similarity in another biometric. The specific blood type for example may selected from any in the group consisting of A, A1, A2, B, B1 and O. Individuals within a subgrouping for example may share a mitochondrial lineage selected from the group consisting of U, X, H, V, T, K and J; Rh D positive or Rh D negative subgroup. Or may share: C positive, C negative, D positive, D negative, E positive and E negative; Cc, Dd and Ee,

Individuals in a subgroup may share a mitochondrial group selected from the group consisting of A, B, C, D, F, G, H, I, J, K, L, M, N, U, V, W, and X.

Understanding of the complexity of animal metabolism, including mammalian and human metabolism reveals that optimizing or enhancing activity can at different times under different circumstances include a variety of paths including, but not limited to: upregulating or down regulating mitochondrial activity, altering or changing mitochondrial location, changing mitochondrial distribution, improving mitochondrial integrity or quality, changes mitochondrial number by increasing or decreasing mitochondrial number, altering mitochondrial dynamics (movement of mitochondria including size and shape change). Mitochondrial dynamics also include the moving of mitochondria within a cell whether or not the movement is associated with a size or shape change. The movement may be antegrade or retrograde for any individual mitochondrion.

The optimization process by definition will result in some improvement. The factor or circumstance being improved will be considered suboptimal, and accordingly will be considered to be deficient in some manner thus comprising a deficit. To show improvement the deficit must be detectable in some manner of observation. The detectable deficit may present in a number of ways including, but not limited to: mitochondrial dysfunction, deficit of cellular metabolism, deficit in individual performance. These deficits may or may not be traceable to a genetic component aberration which as illustrated above may be cellular or mitochondrial. And in some cases may result from a mismatch of cellular and mtDNA. Deficits may result from or be associated with many factors including, but not limited to: a pharmacologic event or course of treatment; a behavior factor, e.g., a sports injury, exposure to extreme temperature, bulimia, anorexia, binging; contacting a toxin; planned or unplanned toxic event; infection; immunologic response; autoimmune episode; inherited deficit; deficit caused by mutation; deficit acquired by one or more life events; and deficits that might be acquired or exacerbated in a secondary fashion due to or due to treatment for another medical condition.

Improved mitochondrial function may manifest in many observable ways including, but not limited to: oxidative phosphorylation energy versus heat production (coupling efficiency) that may be increased or decreased depending on circumstance, free radical generation, free radical scavenging, initiation of apoptosis, mtDNA transcription, mtDNA maintenance including restorative maintenance following injury or medical treatment, mtDNA maintenance wherein the cell or organism has been impacted by a cancer, mitochondrial protein translation, post translational modification, mitochondrial protein import or translocation, ion import or homeostasis, nucleotide translocation, ATP translocation, mitochondrial fission, mitochondrial fusion, Ca⁺⁺ compartmentalization or homeostasis, steroid biosynthesis, the urea cycle, fatty acid oxidation, the tricarboxylic acid cycle, pyruvate metabolism, cellular redox balance, synthesis of precursor compounds including for example a myelin precursor, altering iron metabolism, altering oxygen use, a component or activity of the electron transport chain, an epigenetic modification. These options are not to be considered limiting examples, for example, ion import or homeostasis may involve one or more ions including, but not limited to: calcium, potassium, hydrogen, magnesium, sodium, inorganic ions, etc. and epigenetic includes, but is not limited to: methylation, demethylation, acetylation, histone modification, etc.

Any of these methods may include embodiments comprising a mitochondrial regulatory substance including, but not limited to: rotenone, antimycin, cyanide, amytal, azide, 2, 4-dinitrophenol, oligoymycin and malonate and uncoupling agent including, but not limited to: 2, 4-dinitrophenol (DNP), carbonyl cyanide p-[trifluoromethoxy]-phenyl-hydrazone (FCCP) and oligomycin.

Methods of the present invention include, but are not limited to: embodiments of an individual's or patient's assessment of a medical condition that might be corrected or improved, metabolic status, etc. Assessments of various types are known in the art and are continuously developed by skilled or unskilled artisans in response to a particular issue. Practicing the present invention therefore may involve assessments including, but not limited to assessments of cellular metabolism, mitochondrial function or activity, etc.

A patient's metabolic status may involve consideration of results from sources including, but not limited to: data compiled from a group relevant to a patient, a battery of tests completed by said patient, correlating a test of a biologic trait or a combination of biologic traits with an assessment of clinical improvement, etc. Metabolic status may be elucidated by assessing the patient's metabolic status by assigning at least one value to a biochemical or biophysical observation and/or assigning at least one value to a clinical observation. These or other values or data may be correlated to one another or multiply correlated. Machine learning may be used to aid optimization.

The present invention may be performed at several levels of rigor. At its basic simplicity is a recognition that any organism's metabolism is a complex interconnection of pathways (sequences of biochemical events). Recognizing that there are literally thousands of pathways, some parallel, some opposite, some reversible, most serial, some redundant, etc. each individual living organism, including each human organism will at any time have many activated and many inactivated. Actual pathways in use are not individualized to the organism, but are in constant flux as time and conditions change. Within the organism different tissues or organs will display differentiated pathways, the activity of each varying with time and conditions. Therefore to balance, restore, optimize and/or enhance cellular metabolism that is deficient, compromised or otherwise sub-optimal, the individual must be assessed to determine areas where improvement must be targeted. This application includes a volume of background discussion of multiple factors to be considered when planning to balance, restore, optimize and/or enhance metabolism. Since energy metabolism is a foundation of any organism's activity the discussion was tilted in that respect. And since mitochondria are considered by many to be the metabolic engine of eukaryotic life and several disease states are known to involve compromised energy metabolism, special emphasis is placed on improving mitochondrial function.

In order to balance, restore, optimize and/or enhance cellular metabolism at least one factor in cellular metabolism must be targeted. The practitioner therefore will focus on at least one area for improvement. Different individuals will present with different circumstances. In some more simplistic embodiments. Identification of a disease will be quite instructive. For example, the disease may be known to strongly correlate with a detectable deficit in cellular metabolism. It may be unnecessary as a first pass in these circumstances to obtain tissue samples from that individual. An initial improvement protocol can commence based on disease identification alone. As treatment continues, various attributes of the individual can be detected (monitored) and used for continuing the method. A database or table associating the disease and recommended first actions to take may be helpful for balancing, restoring, optimizing and/or enhancing cellular metabolism in an individual associated with the disease or condition.

At a second level, an individual's genomic content (nuclear and/or mitochondrial) may be known absent functional testing to assess the individual. But if experience has shown that a specific gene or association of genes correlates highly with a present or future detectable deficit, the process of balancing, restoring, optimizing and/or enhancing cellular metabolism might commence on this basis alone.

However, all individuals are not so simple. In advanced practice of this invention multiple observations are contemplated, perhaps, behavioral, morphological, or other easily observed characteristic, but more often and more preferable for higher functioning of this invention, multiple factors will be assessed. Most of the factors discussed are candidates for inclusion as factors to be considered the end goal of balancing, restoring, optimizing and/or enhancing cellular metabolism. Many other factors, that space, brevity consideration and time did not permit inclusion in the present discussion might alternatively or also be considered in the method balancing, restoring, optimizing and/or enhancing cellular metabolism.

As seen above, optimal practice of the present invention is very involved and complex. Each individual will present with a unique set of conditions. Literally thousands of pathways would be expected to be involved in how the individual presents. While modulating any one component or pathway may be a start of optimization, preferably a more robust method will be employed to facilitate more optimal function. Additionally, the individual is not static, any method designed to improve metabolism is expected to trigger change. Continual improvement/optimization preferably employs periodic assessment and reassessing the optimization protocol.

Best practices are beyond human ability. A very preferred embodiment of this invention therefore involves mechanized analysis, self-referencing libraries, preferably using an advanced computing system, and more preferably an advanced computing system periodically or continuously updating self-referencing servers connected to a cloud based network. With this tool in the background, the person or team practicing the present invention will be able to operate at a high level with great benefit to the individual whose metabolism is restored, optimized and/or enhanced. The system can build on previous optimization protocols and results observed therefrom. Accordingly, each iteration, as an individual's progress is monitored and changes are made, and results from multiple individual are incorporated into the advanced computing system, the system will function as a robust, perhaps almost requisite tool for practicing the present invention at its elevated level.

Any of these methods may include embodiments comprising a correlation factor used that might be used in: a) reassessing said patient's condition and metabolic status for use in modifying the earlier protocol and then continuing a treatment using a modified protocol. Assessment may be empirical or may be obtained by questionnaire with answers obtained from an individual, an individual's surrogate, a clinician or other relevant party including authority figure, such as law enforcement, scholastic, employer or relative of the individual.

While the invention may be most applicable to a human person, many applications may be found in veterinary and agriculture arts. As discussed above, energy metabolism is necessary for all cell function. Every chemical change or movement within the cell requires transfer of energy. Heretofore, although energy is a known component of cell physiology and the mitochondrion is known as the organelle integral to electron transport and production of the energy powerhouse ATP, correction of energy anomalies as a part of therapies for other deficiencies has not been appreciated. The present invention addresses this oversight or deficiency and thereby is available to tremendously improve many medicinal therapies.

### REFERENCES

Di Mauro, S., Mitochondrial Medicine, 7-9 2006
Herrmann et al. 1989
Schaart et al. 1989.
Rappaport et al. 1998.
Published online 2013 Apr. 22. doi: 10.3389/fphys.2013.00082
PMCID: PMC3631707
EP2101808A2
Alberio et al., 2007;
Chinnery and Zeviani, 2008;
Spinazzola and Zeviani, 2009
Kay et al. 1997,
Capetanaki, 2002
Nicholls and Budd, 2000
Tolstonog et al., 2001b
Uttam et al., 1996
Gilbert et al., 2001
Brownlees et al., 2002
http://nautil.us/issue/15/turbulence/fruits-and-vegetables-are-trying-to-kill-you
http://www.nature.com/nature/journal/v414/n6865/fig_tab/414807a_F1.html
Istvan R. Boldogh and Liza A. Pon.
[http://www.sciencedirect.com/science/article/pii/S0167488906000486
NEJM 2012; 366: 1132-41, supplementary appendix
http://www.mdpi.com/metabolites/metabolites-04-00831/article_jeploy/htnd/images/metabolites-04-00831-g001-1024.png
www.itsokaytobesmart.comn

1) An improved method of medical therapy, the improvement comprising administering a medicament selected to balance, restore, optimize and/or enhance cellular metabolism that is deficient, compromised or otherwise determined to be sub-optimal, said deficient, compromised or otherwise sub-optimal function resulting from an event selected from the group consisting of i) a detectable deficit in cellular metabolism, ii) a condition that benefits from therapeutic intervention, iii) the therapeutic intervention, and a condition benefiting from rebalancing metabolism of selected cells or cell types.

2) The improved method according to claim 1 wherein cellular metabolism is restored by improving mitochondrial quantity and/or activities.

3) The improved method according to claim 2 wherein cellular metabolism is restored by activating or by inhibiting mitochondrial biogenesis.

4) The improved method according to claim 2 wherein cellular metabolism is restored by inhibiting or enhancing mitochondrial degradation (mitophagy).

5) The improved method according to claim 1 wherein cellular metabolism is restored by enhancing mitochondrial stability.

6) The improved method according to claim 1 wherein cellular metabolism is restored by increasing or decreasing mitochondrial quantity.

7) The improved method according to claim 1 wherein cellular metabolism is restored by altering mitochondrial location in a cell.

8) The improved method according to claim 1 wherein cellular metabolism is restored, optimized or enhanced by improving mitochondrial tissue distribution.

9) The improved method according to claim 1 wherein cellular metabolism is restored, optimized or enhanced by improving one or more mitochondrial function or activity.

10) The improved method according to claim 9 wherein the improved mitochondrial function or activity comprises one or more consideration selected from the group consisting of: oxidative phosphorylation, coupling efficiency (energy versus heat production), free radical generation, free radical scavenging, initiation of apoptosis, mtDNA transcription, mtDNA maintenance, generation of reactive oxygen species, controlling DNA acetylation, controlling DNA methylation, histone modification, mitochondrial protein translation, post translational modification or mitochondrial proteins, mitochondrial protein import or translocation, ion import, ion homeostasis, permeability to one or more ions, transmembrane potential, nucleotide translocation, ATP translocation, mitochondrial fission, mitochondrial fusion, Ca⁺⁺ compartmentalization or homeostasis, steroid biosynthesis, a component of the urea cycle, fatty acid oxidation, a component of the tricarboxylic acid cycle, pyruvate metabolism, cellular redox balance, synthesis of precursor compounds for a mitochondrial function or activity, iron metabolism, oxygen metabolism and any component or activity of the electron transport chain.

11) A method of improving medical treatment, said method comprising:
i. assessing deficient or altered energy metabolism associated with a medical treatment;
ii. administering to a recipient of said treatment a substance that optimizes or enhances mitochondrial capacity and/or performance with the result that dysfunctional cellular energy metabolism is reduced or ameliorated.

12) The improved method according to claim 11 wherein mitochondrial dysfunction is reduced or ameliorated following, with relation to i. obtaining a biosubstance from said recipient.

13) A method for optimization of medical treatment, said method comprising:
i) assessing a patient to determine a medical condition to be corrected or improved
ii) assessing the patient's metabolic status;
iii) selecting or designing a protocol to improve or optimize the patient's metabolic status;
iv) initiating the treatment including the protocol of iii).

14) The method according to claim 13 further comprising:
a) reassessing said patient's condition and/or metabolic status;
b) modifying the protocol based on a); and
c) continuing treatment including the modified protocol.

15) The method according to claim 13 wherein the metabolic status assessment comprises assessing cellular metabolism.

16) The method according to claim 13 wherein the metabolic status assessment comprises assessing mitochondrial function or activity.

17) The method according to claim 13 wherein the assessing patient's metabolic status uses data compiled from a group relevant to said patient.

18) The method according to claim 13 wherein the metabolic status assessment comprises a battery of tests completed by said patient.

19) The method according to claim 13 wherein the metabolic status assessment comprises correlating a test of a biologic trait or a combination of biologic traits with an assessment of clinical improvement.

20) The method according to claim 19 wherein the correlation is a factor used in:
a) reassessing said patient's condition and metabolic status;
b) modifying the protocol based on a); and
c) continuing treatment including the modified protocol.
